(19) United States
(12) Patent Application Publication
(10) Pub. No.: US 2026/0114972 A1
(43) Pub. Date: Apr. 30, 2026

(54) SYSTEMS AND METHODS FOR PREDICTING PERIODONTAL POCKET DEPTH AND/OR OTHER INTRAORAL CONDITIONS

(71) Applicant: Align Technology, Inc., San Jose, CA (US)

(72) Inventors: Shai Farkash, Hod Hasharon (IL); Ran Katz, Hod Hasharon (IL); Maayan Moshe, Ramat Hasharon (IL); Ran Feder, Herzliya (IL); Moti Ben-Dov, Tel Mond (IL)

(21) Appl. No.: 19/369,787

(22) Filed: Oct. 27, 2025

Related U.S. Application Data
(60) Provisional application No. 63/712,757, filed on Oct. 28, 2024.

(51) Int. Cl. A61C 19/04; A61B 5/00

(52) U.S. Cl. CPC A61C 19/043; A61B 5/0088; A61B 5/7264; A61B 5/7275

(57) ABSTRACT

Systems and methods for evaluating a patient's intraoral health are provided. In some embodiments, a method includes receiving scan data of a patient's intraoral cavity; receiving additional data for the patient, the additional data being different from the scan data; determining a first prediction of a condition of the intraoral cavity based on the scan data; determining a second prediction of a condition of the intraoral cavity based on the additional data; generating a predicted condition for the intraoral cavity based on a combination of the first and second predictions; and outputting an indication of the predicted condition on a display.

### CROSS-REFERENCE TO RELATED APPLICATION(S)

The present application claims the benefit of priority to U.S. Provisional Application No. 63/712,757, filed Oct. 28, 2024, which is incorporated by reference herein in its entirety.

### TECHNICAL FIELD

The present technology generally relates to dentistry, and in particular, to systems and methods for predicting periodontal pocket depth and/or other intraoral conditions.

### BACKGROUND

Periodontal disease (also known as periodontitis) involves inflammation and infection of the tissues supporting the teeth, such as the gingiva, ligaments, and bone. In the early stages of periodontal disease, the gingiva become inflamed (gingivitis) and may pull away from the tooth, leading to formation of periodontal pockets. In the advanced stages of periodontal disease, the tissues supporting the tooth may erode, leading to loosening or even loss of the tooth. Disease progression may be prevented through early detection and intervention, but it may be difficult for individuals to determine whether they are at risk of periodontal disease, particularly since noticeable symptoms may not appear until the later stages of the disease. Conventional techniques for diagnosing periodontal disease generally rely upon visual examination and periodontal probing to evaluate periodontal pocket depth, but such techniques may not be reliable for detecting early-stage disease and/or obscured disease sites.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIGS. 1A-1D illustrate progression of periodontal disease with a corresponding increase in periodontal pocket depth.

FIG. 2 is a schematic block diagram illustrating a workflow for training a periodontal parameter prediction model, in accordance with embodiments of the present technology.

FIG. 3A illustrates a mesh model of a patient's dental arch generated from intraoral scan data, in accordance with embodiments of the present technology.

FIG. 3B illustrates a color image of a patient's teeth and gingiva obtained via intraoral scanning, in accordance with embodiments of the present technology.

FIG. 3C illustrates a texture map of a portion of a patient's dental arch generated from intraoral scan data, in accordance with embodiments of the present technology.

FIG. 3D illustrates a NIR image of a patient's teeth and gingiva obtained via intraoral scanning, in accordance with embodiments of the present technology.

FIG. 4 is a flow diagram illustrating a method for predicting a periodontal parameter, in accordance with embodiments of the present technology.

FIG. 5A illustrates a 3D model of a dental arch with segmented gingival regions, in accordance with embodiments of the present technology.

FIG. 5B illustrates 2D image data of a gingival region, in accordance with embodiments of the present technology.

FIG. 6 is a schematic block diagram illustrating a workflow for evaluating a patient's intraoral health using a periodontal parameter prediction model, in accordance with embodiments of the present technology.

FIG. 7A is a flow diagram illustrating a method for evaluating a patient's intraoral health, in accordance with embodiments of the present technology.

FIG. 7B illustrates a user interface for displaying predicted periodontal parameters of a patient, in accordance with embodiments of the present technology.

FIG. 7C illustrates another user interface for displaying predicted periodontal parameters of a patient, in accordance with embodiments of the present technology.

FIG. 8 is a schematic block diagram illustrating a workflow for evaluating a patient's intraoral health using a periodontal parameter prediction model, in accordance with embodiments of the present technology.

FIG. 9 is a flow diagram illustrating a method for evaluating a patient's intraoral health, in accordance with embodiments of the present technology.

FIG. 10 is a flow diagram illustrating a method for generating a patient-specific periodontal parameter prediction model, in accordance with embodiments of the present technology.

FIG. 11 is a flow diagram illustrating a method for evaluating a patient's intraoral health, in accordance with embodiments of the present technology.

FIG. 12A is a schematic block diagram illustrating a workflow for determining a periodontal pocket depth value, in accordance with embodiments of the present technology.

FIG. 12B is a schematic block diagram illustrating a workflow for identifying causation factors from patient data, in accordance with embodiments of the present technology.

FIG. 12C is a schematic block diagram illustrating a workflow for determining potential causes of abnormal periodontal pocket depth values based on causation factors, in accordance with embodiments of the present technology.

FIG. 13 is a flow diagram illustrating a method for evaluating a patient's intraoral health, in accordance with embodiments of the present technology.

FIG. 14A is a schematic block diagram illustrating a workflow for predicting a condition of a patient's intraoral cavity, in accordance with embodiments of the present technology.

FIG. 14B is a schematic block diagram illustrating a workflow for predicting a condition of a patient's intraoral cavity, in accordance with embodiments of the present technology.

FIG. 15A schematically illustrates a system for performing intraoral scanning and/or generating 3D digital representations of a patient's intraoral cavity, in accordance with embodiments of the present technology.

FIG. 15B is a partially schematic illustration of an example scanner that may be used in the system of FIG. 15A, in accordance with embodiments of the present technology.

### DETAILED DESCRIPTION

The present technology relates to methods for evaluating a patient's intraoral health, and associated systems and devices. In some embodiments, for example, a method includes receiving scan data of a patient's intraoral cavity, and receiving additional data representing one or more patient-specific characteristics of the patient. The additional data may be non-scan data, such as demographic information, health condition data, previous periodontal parameter data, palate color data, other imaging modality data, bone height and/or bone loss data, gum recession data, inflammation data, tooth mobility data, and/or occlusion data. The method can include determining, based on the scan data and additional data, a periodontal parameter indicative of a periodontal condition of the patient, such as a periodontal pocket depth value for one or more of the patient's teeth. The periodontal parameter may be determined using a machine learning model that is trained on scan data and periodontal parameter measurements from a plurality of patients. In some embodiments, the machine learning model is configured to use the additional data to account for the one or more patient-specific characteristics when determining the periodontal parameter, thereby generating a prediction of the periodontal parameter that is normalized to the patient-specific characteristics (e.g., soft tissue color, previous periodontal pocket depth values). The method can continue with outputting an indication of the periodontal parameter on a display.

As another example, a method for evaluating a patient's intraoral health can include receiving scan data of a patient's intraoral cavity and determining, based on the scan data, a periodontal pocket depth value for the patient, where the periodontal pocket depth value is determined using a machine learning model that is trained on scan data and periodontal parameter measurements from a plurality of patients. The method can include identifying a potential cause of the periodontal pocket depth value. For example, the scan data and/or additional data can be analyzed to detect one or more causation factors (e.g., calculus build up, increased tooth wear, increased occlusal contacts, bite force, frequency of deep periodontal pocket depths, distribution of deep periodontal pocket depths, blood pressure, diabetes status, smoking status), and the potential cause can be identified based on the causation factors. The method can include outputting an indication of the periodontal pocket depth and the potential cause on a display and, optionally, a treatment recommendation.

In a further example, a method for evaluating a patient's intraoral health can include receiving scan data of a patient's intraoral cavity, and receiving additional data for the patient, the additional data being associated with a different measurement modality than the scan data. For example, the additional data can include x-ray data, bone height and/or bone loss data, gum recession data, inflammation data, tooth mobility data, and/or occlusion data. The method can include detecting, based on the scan data and the additional data, a condition of the patient's intraoral cavity (e.g., periodontal disease, a deep periodontal pocket, bruxism, tooth decay, dental plaque, an oral health metric), and outputting an indication of the detected condition on a display.

The present technology can provide many advantages compared to conventional approaches for evaluating a patient's intraoral health. For example, conventional methods typically use a physical probe to measure periodontal pocket depth, but this technique may be time-consuming and uncomfortable for the patient, and may require a trained dental practitioner to perform. Intraoral scans of a patient's teeth and gums may provide a non-intrusive alternative for measuring periodontal pocket depth, but patient-specific variations in soft tissue color, gum shape, bone characteristics, etc., may affect the topography and visual appearance of the gingival tissue in scan data, such that it may be difficult to reliably determine pocket depth value based on scan data alone.

The present technology can address these and other challenges by using scan data in combination with other types of data to quickly and accurately evaluate periodontal pocket depth and/or other intraoral conditions. For instance, intraoral scans can be analyzed to provide a preliminary assessment of periodontal pocket depth values, e.g., to aid the clinician in determining whether full probing of the patient is indicated. In some embodiments, additional data is used to “normalize” the predicted periodontal pocket depth value to account for patient-specific characteristics, thereby providing improved reliability compared to approaches that use scan data alone and/or use generic algorithms that are not customized to the particular patient. Moreover, the techniques herein can be used to identify the underlying causes of abnormal periodontal pocket depth values to provide guidance for patient treatment. Furthermore, the techniques herein can be used to detect other intraoral conditions besides periodontal pocket depth, such as bruxism, tooth decay, dental plaque, etc., thereby providing a flexible platform for diagnosis and monitoring of many different types of dental and periodontal diseases.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” “lower,” “left,” “right,” etc., can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Detection of Intraoral Conditions

The present technology provides methods and systems for determining whether a patient has an intraoral condition, such as periodontal disease, gingivitis, tooth decay, dental plaque, bruxism, etc. For example, certain intraoral conditions may be correlated to changes in the color and/or shape of the teeth, gingiva, bone, and/or other tissues of the intraoral cavity. Such changes may be captured using scan data and/or additional data obtained using other measurement modalities, and the scan data and/or additional data may be analyzed using software algorithms (e.g., trained machine learning models, rule-based algorithms) to provide automated detection of various intraoral conditions.

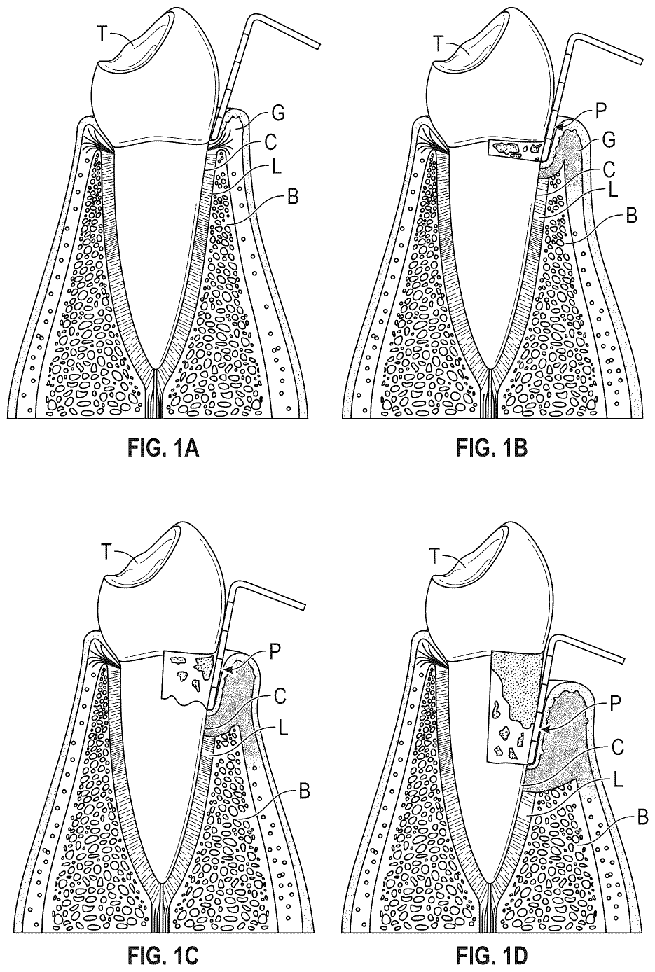

FIGS. 1A-1D illustrate progression of periodontal disease with a corresponding increase in periodontal pocket depth. Referring first to FIG. 1A, a tooth T with healthy supporting tissue, such as gingiva G, cementum C, ligaments L (e.g., a periodontal ligament (PDL)), and bone B, is shown. Referring next to FIG. 1B, poor oral hygiene may result in buildup of bacterial plaque on the tooth T, leading to inflammation of the gingiva G. The gingiva G may pull away from the tooth T, thus forming a periodontal pocket P. Minor erosion of the cementum C, ligaments L, and/or bone B may also occur at this stage. Referring next to FIG. 1C, as the periodontal disease progresses, the periodontal pocket P may deepen, allowing bacteria to invade deeper into the tissue and causing further loss of the cementum C, ligaments L, and/or bone B. Referring next to FIG. 1D, in advanced periodontal disease, significant deepening of the periodontal pocket P and loss of the cementum C, ligaments L, and/or bone B has occurred, which may lead to loosening or even loss of the tooth T. Frequent monitoring of the depth of the periodontal pocket P over time may be advantageous for detecting the onset of periodontal disease at an early stage and implementing appropriate therapeutic interventions (e.g., improvements in oral hygiene practices), thereby slowing or preventing disease progression.

### A. Prediction of Periodontal Parameters

In some embodiments, the present technology provides methods for predicting periodontal parameters of a patient, based at least in part on scan data of the patient's intraoral cavity. This approach advantageously allows for evaluation of periodontal pocket depth and/or other characteristics of the tissues supporting a patient's tooth without requiring physical probing of the intraoral cavity. The evaluation may be performed using machine learning models that are trained on scan data to learn the correlations between features of intraoral structures depicted in the scan data (e.g., topography, color) and periodontal parameters of such structures.

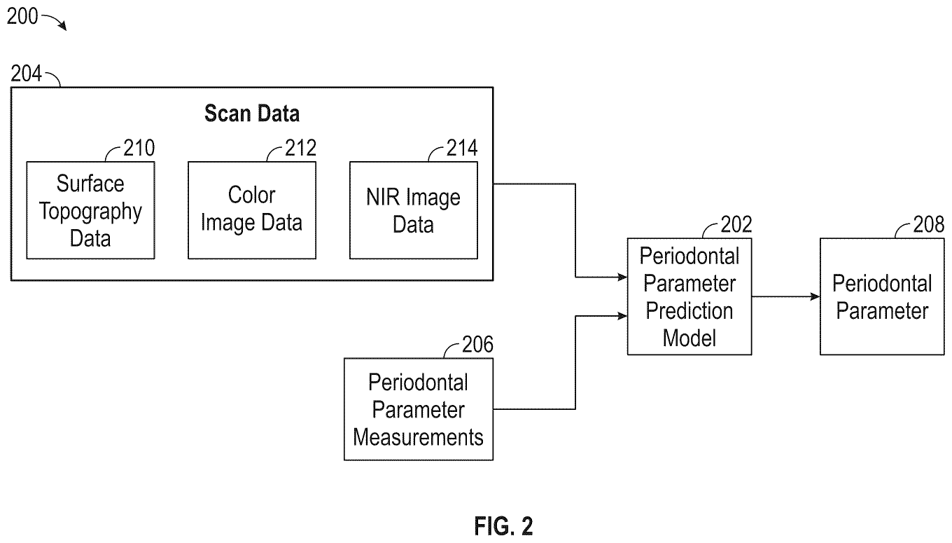

FIG. 2 is a schematic block diagram illustrating a workflow 200 for training a periodontal parameter prediction model 202, in accordance with embodiments of the present technology. The workflow 200 can be implemented using any of the systems and devices described herein. In some embodiments, some or all of the processes of the workflow 200 are implemented as computer-readable instructions (e.g., program code) that are configured to be executed by one or more processors of a computing device (e.g., a client device, a server device, or suitable combinations thereof).

The periodontal parameter prediction model 202 is a machine learning model that is trained on scan data 204 and on periodontal parameter measurements 206 to predict a periodontal parameter 208 for one or more of a patient's teeth. The periodontal parameter 208 can be a quantitative or qualitive indication of the condition of one or more intraoral tissues proximate to and/or supporting a tooth, such as the gingiva, cementum, ligaments, bone, etc. The periodontal parameter 208 can be a number, score, measurement, descriptor, classification, etc. For example, the periodontal parameter 208 can be a periodontal pocket depth value indicating the depth of a periodontal pocket proximate to a tooth (e.g., in mm). Pocket depth values may be determined for one or more different locations along a tooth, such as at a distal location, a mesial location, a middle location (e.g., at or near the centerline of the tooth), a buccal location, or a lingual location. As another example, the periodontal parameter 208 can be a gingival index (GI) or a modified gingival index (MGI) representing the condition of the gingiva (e.g., 0=normal gingiva, 1=mild inflammation, 2=moderate inflammation, 3=severe inflammation). Other periodontal parameters 208 that may be predicted include gingival margin level (e.g., distance of the gingival margin relative to the cemento-enamel junction (CEJ)), clinical attachment loss (e.g., distance of the CEJ to the depth of the periodontal pocket), bleeding on probing, furcation involvement, tooth mobility index (e.g., Miller index), plaque index, radiographic bone loss, overall oral hygiene status, and/or presence of crowded and/or maloccluded teeth.

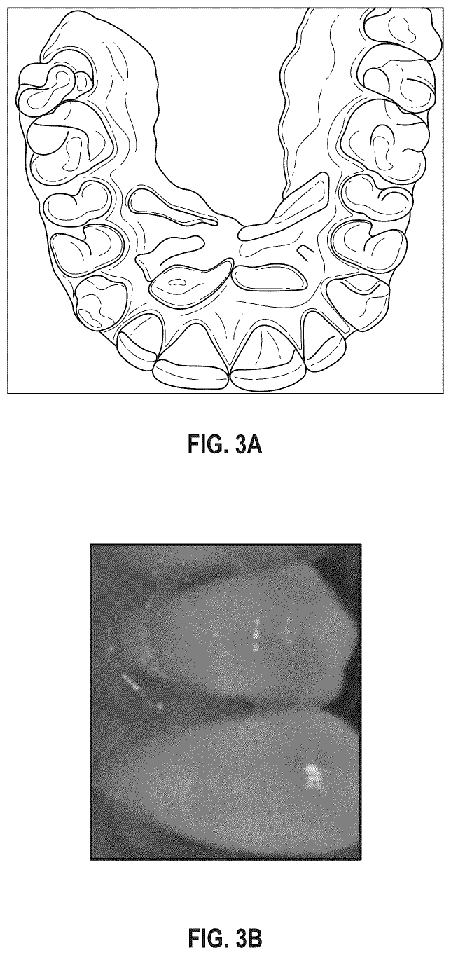

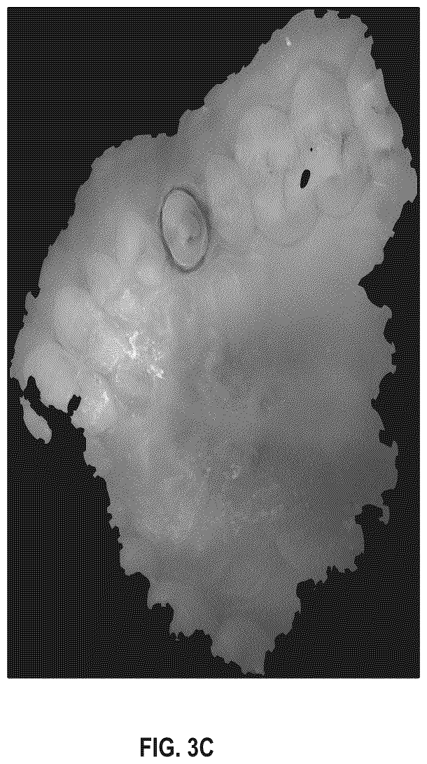

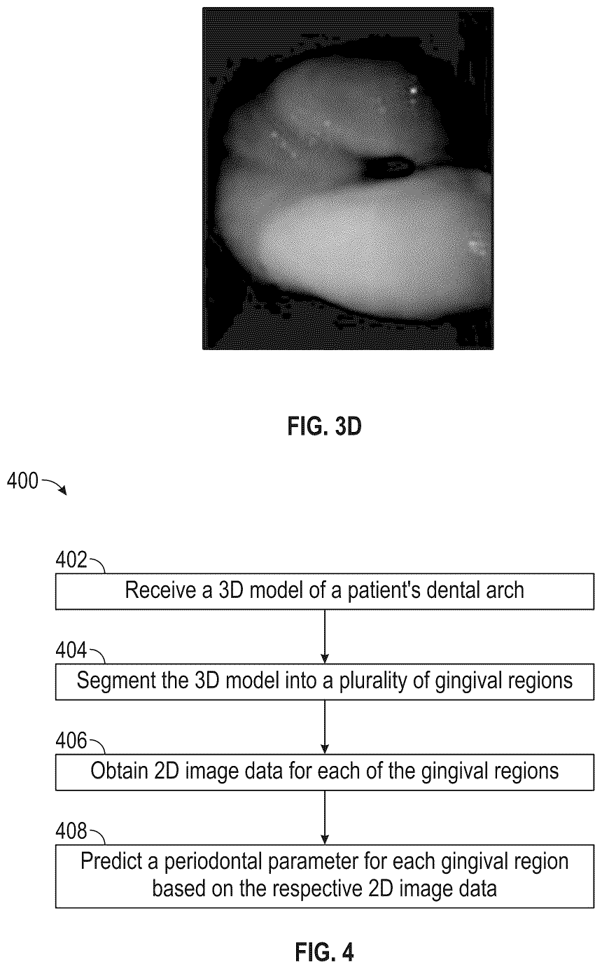

The scan data 204 can depict one or more intraoral structures, such as the teeth, gingiva, palate, tongue, cheeks, etc. The scan data 204 can be obtained using an intraoral scanning system, such as any of the systems described in Section II below. The scan data 204 can include surface topography data 210 that provides a 3D digital representation of the surface topography of the intraoral structures, such as one or more point clouds, height/depth maps, surface models, mesh models, etc. For example, FIG. 3A illustrates a mesh model of a patient's dental arch generated from intraoral scan data, in accordance with embodiments of the present technology.

In some embodiments, the scan data 204 also includes other types of data depicting other 2D and/or 3D characteristics of the intraoral structures. The use of other data types may be advantageous for capturing features of the intraoral structures that may be used to evaluate intraoral health but may not be apparent from the surface topography data 210 alone. For instance, the scan data 204 can include color image data 212, such as one or more photographs, videos, etc., depicting the color of the intraoral structures. As an example, FIG. 3B illustrates a color image of a patient's teeth and gingiva obtained via intraoral scanning, in accordance with embodiments of the present technology.

Optionally, the color image data 212 can include or be processed to include a continuous texture map that provides a 2D “flattened” representation of the color of a portion of a dental arch or an entire dental arch. For example, FIG. 3C illustrates a texture map of a portion of a patient's dental arch generated from intraoral scan data, in accordance with embodiments of the present technology. The texture map may be generated by aligning and stitching together multiple color images of different regions of the intraoral cavity.

The scan data 204 may include image data obtained at other wavelengths besides visible wavelengths, such as near-infrared (NIR) image data 214, infrared image data, ultraviolet image data, etc. For example, FIG. 3D illustrates a NIR image of a patient's teeth and gingiva obtained via intraoral scanning, in accordance with embodiments of the present technology. In some embodiments, the scan data 204 includes fluorescence image data. Fluorescence image data may be obtained by illuminating the intraoral cavity with light (e.g., emitted from the scanner or other light source) at one or more excitation wavelengths, and fluorescent images may be obtained at one or more emission wavelengths. For instance, red fluorescence (e.g., excitation wavelengths greater than 655 nm) may be used to image calculus on the teeth, while blue fluorescence (e.g., wavelengths within a range from 400 nm to 450 nm) may be used to image bacteria on the teeth.

In some embodiments, scan data 204 is obtained for a plurality of patients, and the scan data 204 for each patient can labeled with or otherwise associated with the actual periodontal parameter measurements 206 for the particular patient. In some embodiments, the periodontal parameter measurements 206 are obtained from a periodontal chart or other dental record for the patient that lists periodontal pocket depths and/or other periodontal measurements obtained through physical probing and/or visual assessment of the patient's intraoral cavity. The scan data 204 and periodontal parameter measurements 206 across all patients can be compiled into a training data set, and the training data set can be used to train the periodontal parameter prediction model 202, e.g., using supervised learning, unsupervised learning, reinforcement learning, transfer learning, or suitable combinations thereof. Accordingly, the periodontal parameter prediction model 202 can learn how the features depicted in the scan data 204 for a patient correlate to the actual periodontal parameter measurements 206 for the patient.

The periodontal parameter prediction model 202 can utilize any of the following machine learning algorithms: a regression algorithm (e.g., ordinary least squares regression, linear regression, logistic regression, stepwise regression, multivariate adaptive regression splines, locally estimated scatterplot smoothing), an instance-based algorithm (e.g., k-nearest neighbor, learning vector quantization, self-organizing map, locally weighted learning), regularization algorithms (e.g., ridge regression, least absolute shrinkage and selection operator, elastic net, least-angle regression), a decision tree algorithm (e.g., Iterative Dichotomiser 3 (ID3), C4.5, C5.0, classification and regression trees, chi-squared automatic interaction detection, decision stump, M5), a Bayesian algorithm (e.g., naïve Bayes, Gaussian naïve Bayes, multinomial naïve Bayes, averaged one-dependence estimators, Bayesian belief networks, Bayesian networks, hidden Markov models, conditional random fields), a clustering algorithm (e.g., k-means, single-linkage clustering, k-medians, expectation maximization, hierarchical clustering, fuzzy clustering, density-based spatial clustering of applications with noise (DBSCAN), ordering points to identify cluster structure (OPTICS), non-negative matrix factorization (NMF), latent Dirichlet allocation (LDA), Gaussian mixture model (GMM)), an association rule learning algorithm (e.g., apriori algorithm, equivalent class transformation (Eclat) algorithm, frequent pattern (FP) growth), an artificial neural network algorithm (e.g., perceptrons, neural networks, back-propagation, Hopfield networks, autoencoders, Boltzmann machines, restricted Boltzmann machines, spiking neural nets, radial basis function networks), a deep learning algorithm (e.g., deep Boltzmann machines, deep belief networks, convolutional neural networks, stacked auto-encoders), a dimensionality reduction algorithm (e.g., PCA, independent component analysis (ICA), principle component regression (PCR), partial least squares regression (PLSR), Sammon mapping, multidimensional scaling, projection pursuit, linear discriminant analysis, mixture discriminant analysis, quadratic discriminant analysis, flexible discriminant analysis), an ensemble algorithm (e.g., boosting, bootstrapped aggregation, AdaBoost, blending, gradient boosting machines, gradient boosted regression trees, random forest), or suitable combinations thereof. The type and architecture of the machine learning algorithm can be selected based on the type of inputs and outputs for the periodontal parameter prediction model 202, among other considerations.

In some embodiments, for example, the periodontal parameter prediction model 202 is or includes a deep learning neural network, such as a convolutional neural network (CNN). CNNs are a type of machine learning algorithm that can be used in the processing of images and/or other array-like data structures (e.g., tensors). A CNN is composed of a plurality of layers, with each layer including one or more neurons to which the operations described herein are applied. The CNN can transform input data (e.g., data received at an input layer) into output data (e.g., data output by an output layer) through a network architecture including a plurality of intermediate layers. In some embodiments, the plurality of intermediate layers include one or more convolutional layers. Each convolutional layer of a CNN can apply at least one filter (also known as a “kernel”) to input data from a preceding layer via a convolutional operation. The parameters of the kernel (e.g., kernel size, weight, biases, parameters of the kernel function(s)) can be learned from training data (e.g., using backpropagation). The CNN can optionally include multiple convolutional layers, with the input data for each convolutional layer including output data from a preceding layer (e.g., another convolutional layer or another type of layer).

In some embodiments, the CNN includes one or more additional layers besides the one or more convolutional layers, such as at least one pooling layer and/or at least one fully connected layer. The at least one pooling layer can apply a spatial reduction operation to a preceding layer. In some embodiments, the at least one pooling layer performs dimensionality reduction. The at least one pooling layer can apply any variety of operations, such as max pooling, min pooling, average pooling, and global pooling. The at least one fully connected layer is connected to all preceding and succeeding layers. The at least one fully connected layer can apply a transformation to a preceding layer. In some embodiments, the at least one fully connected layer includes a linear transformation (e.g., affine functions). In some embodiments, the at least one fully connected layer includes a non-linear transformation (e.g., sigmoid, softmax, tanh, rectified linear unit functions). While the CNN has been discussed with respect to the plurality of layers, it should be understood that any of the layers can include one or more neurons at which operations are applied. Further, the CNN can include any arrangement of layers forming a customized network architecture. The prediction produced by the CNN can include output data determined from a convolutional layer, pooling layer, fully connected layer, or any other layer of the CNN.

Alternatively or in combination, the periodontal parameter prediction model 202 can use other types of machine learning models to predict the periodontal parameter 208, such as recurrent neural networks (RNNs), generative adversarial networks (GANs), capsule networks (CapsNets), graph neural networks (GNNs), autoencoders, other types of artificial neural networks (ANNs), or any of the other machine learning algorithm types described herein.

FIG. 4 is a flow diagram illustrating a method 400 for predicting a periodontal parameter, in accordance with embodiments of the present technology. The method 400 can be implemented using any of the systems and devices described herein. In some embodiments, some or all of the processes of the method 400 are implemented as computer-readable instructions (e.g., program code) that are configured to be executed by one or more processors of a computing device (e.g., a client device, a server device, or suitable combinations thereof).

The method 400 can begin at block 402 with receiving a 3D model of a patient's dental arch. The 3D model can be any 3D digital representation of the surface topography of one or more intraoral structures of the dental arch (e.g., teeth, gingiva, palate), such as a point cloud, height/depth map, surface model, mesh model, etc. In some embodiments, the 3D model is generated from intraoral scan data of the dental arch. Optionally, the process of block 402 can include receiving scan data of the patient's dental arch, and generating the 3D model based on the scan data.

At block 404, the method 400 can include segmenting the 3D model into a plurality of gingival regions. The gingival regions can include any portion of the gingiva proximate to one or more teeth where a prediction of a periodontal parameter is to be performed. The gingival regions may include a mesial region (e.g., at or proximate to the mesial end of the tooth), a distal region (e.g., at or proximate to the distal end of the tooth), a middle region (e.g., between the mesial and distal ends of the tooth), a buccal region, a lingual region, etc. For instance, in embodiments where the periodontal parameter is periodontal pocket depth, the gingival regions can include locations where periodontal pocket depth values would typically be obtained via probing.

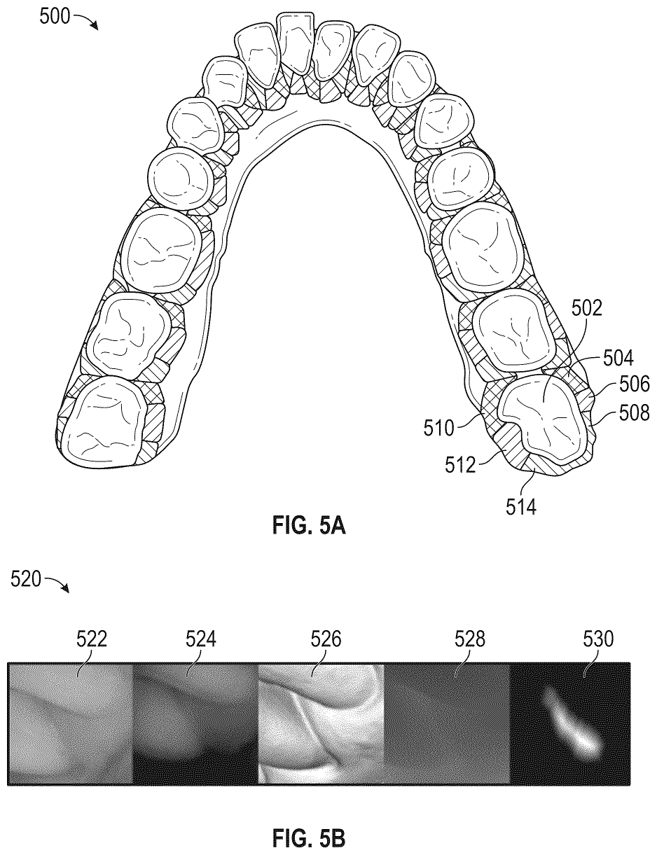

For example, FIG. 5A illustrates a 3D model 500 of a dental arch with segmented gingival regions, in accordance with embodiments of the present technology. As shown in FIG. 5A, the gingiva proximate to each tooth 502 of the model 500 has been segmented into the following regions: a buccal mesial region 504, a buccal middle region 506, a buccal distal region 508, a lingual mesial region 510, a lingual middle region 512, and a lingual distal region 514. In other embodiments, however, the model 500 may be segmented differently. For example, the gingiva proximate to each tooth 502 may be segmented into a single buccal region (e.g., encompassing the buccal mesial region 504, buccal middle region 506, buccal distal region 508) and a single lingual region (e.g., encompassing the lingual mesial region 510, lingual middle region 512, and the lingual distal region 514). Moreover, segmentation can be performed for only a subset of teeth 502 of the model 500, rather than all of the teeth 502 (e.g., if predictions are to be made only for certain teeth 502).

Referring again to FIG. 4, the segmentation process of block 404 can be performed in many different ways. For example, the gingival regions can be segmented using a software algorithm that identifies each tooth in the model for which a prediction is to be made, and then detects regions of the gingiva proximate to each identified tooth that meet certain segmentation criteria. The segmentation criteria can include whether a particular point is within a certain distance of the tooth surface (e.g., the lingual surface of buccal surface), whether a particular point is within a certain distance with respect to a reference location on the tooth (e.g., the distal end of the tooth, the mesial end of the tooth, the centerline of the tooth), etc. In some embodiments, relevant gingival regions are identified using nearest-neighbor distance and clustering.

At block 406, the method 400 can include obtaining 2D image data for each of the gingival regions. The 2D image data can be obtained from scan data of the patient's dental arch, which may or may not be the same as the data used to generate the 3D model of the dental arch. The scan data can include surface topography data, color image data, NIR image data, and/or any other data obtained via intraoral scanning, e.g., as discussed above with respect to FIG. 2. In some embodiments, the scan data depicts a relatively large portion of the dental arch spanning a plurality of gingival regions and/or is in a 3D format (e.g., a 3D digital model), and the 2D image data depicts an individual gingival region for which a prediction is to be made. For instance, the 2D image data can include one or more 2D images in which the gingival region of interest is located at or near the center of each image. In some embodiments, the 2D image data does not show any other gingival regions besides the gingival region of interest, or the other gingival regions may be located at the periphery of the image rather than the center.

The 2D image data may be obtained by sampling a subset of the scan data and/or converting the scan data into a 2D format. For example, in embodiments where the scan data includes image data, such as color image data and/or NIR image data, the process of block 406 can include selecting at least one image that shows the gingival region of interest. Optionally, the image may be adjusted (e.g., cropped, rotated, translated) so that the gingival region is substantially centered in the image and/or so that other gingival regions are minimally visible or not visible in the image. In some embodiments, the image may be segmented to isolate the gingival region of interest from the other structures depicted in the image, and subsequent analysis may be performed on the segmented gingival region only.

For surface topography data, the process of block 406 can include generating an image of the portion of the surface topography data depicting the gingival region of interest. For instance, in embodiments where the surface topography data is provided as a 3D digital model (e.g., a mesh model), an image of the 3D digital model can be generated using virtual camera parameters (e.g., camera position and/or orientation) that are selected so that the gingival region is substantially centered in the image and/or so that other gingival regions are minimally visible or not visible in the image. The virtual camera parameters can be based on pose data of the intraoral scanner during the scanning procedure (e.g., the tracked position and/or orientation of the scanner relative to the teeth). The image of the 3D digital model can provide a 2D representation of relevant topographical information, such as distances/heights, surface normals, segmentations, etc. For example, the image of the 3D digital representation can be a grayscale or heatmap image in which different grayscale or heatmap values represent different depth/height values. Optionally, the image may be adjusted (e.g., cropped, rotated, translated) so that the gingival region is substantially centered in the image and/or so that other gingival regions are minimally visible or not visible in the image. In some embodiments, the image may be segmented to isolate the gingival region of interest from the other structures depicted in the image, and subsequent analysis may be performed on the segmented gingival region only.

In some embodiments, the 2D image data for each gingival region includes a plurality of 2D images showing the gingival region, where each 2D image is associated with a different imaging modality. For example, the 2D image data can include a first 2D image that is generated from surface topography data of the gingival region, a second 2D image that is generated from color image data of the gingival region, a third 2D image that is generated from NIR image data of the gingival region, etc. Each 2D image (which may also be referred to herein as a “channel”) can provide different types of information relevant to predicting the periodontal parameter.

FIG. 5B illustrates 2D image data 520 of a gingival region, in accordance with embodiments of the present technology. In the illustrated embodiment, the 2D image data 520 includes a plurality of different 2D images for the gingival region, with each image corresponding to a different image modality: a color image 522, an NIR image 524, a topography image 526, a depth map image 528, and a segmentation image 530 depicting the tooth-gingiva junction. In other embodiments, however, some of the image modalities shown in FIG. 5B may be omitted and/or the 2D image data 520 may include data from other types of image modalities (e.g., ultraviolet images).

Referring again to FIG. 4, at block 408, the method 400 can include predicting a periodontal parameter for each gingival region based on the respective 2D image data. The periodontal parameter can be a quantitative or qualitive indication of the condition of one or more intraoral tissues proximate to and/or supporting a tooth, such as a periodontal pocket depth value, GI, MGI, etc. The periodontal parameter may be predicted using a trained machine learning model (e.g., the periodontal parameter prediction model 202 of FIG. 2). For example, a CNN can be trained to analyze the 2D image data for each gingival region and to predict the periodontal parameter for that gingival region. As discussed elsewhere herein, the CNN can be trained on scan data and periodontal parameter measurements from a plurality of patients. In some embodiments, the CNN is a residual neural network (e.g., ResNet50), and training is performed using mean-square-log-error loss for pocket depth value regression. The input to the CNN can be the 2D image data for a particular gingival region and the output of the CNN can be a prediction of the periodontal parameter for that gingival region.

Each predicted periodontal parameter may optionally be associated with a confidence score, which may be a numerical value (e.g., a percentage or probability value), a quantitative assessment (e.g., “high confidence,” “low confidence”), or a combination thereof that represents the estimated degree of accuracy of the prediction. The confidence score may be produced by the same algorithm that predicts the periodontal parameter (e.g., the periodontal parameter prediction model 202 of FIG. 2).

In some embodiments, the process of block 408 includes predicting a single periodontal parameter for an individual gingival region based on the 2D image data for that gingival region. Alternatively, the process of block 408 can include predicting multiple periodontal parameters for an individual gingival region (e.g., based on different subsets of the 2D image data for that gingival region), then combining (e.g., averaging) the predictions to obtain an aggregate prediction. Optionally, the process of block 408 can include generating predictions for a region of interest larger than an individual gingival region, such as a periodontal parameter for a particular side of a tooth (e.g., buccal side, lingual side), for a particular tooth, for a particular group of teeth (e.g., sextant, quadrant), or for an entire dental arch. The prediction for a larger region of interest can be any combination, statistic, etc., calculated from the predictions for the individual gingival regions, such as a mean predicted periodontal parameter (e.g., mean periodontal pocket depth value), a maximum predicted periodontal parameter (e.g., maximum periodontal pocket depth value), etc.

### B. Patient-Specific Normalization

In some embodiments, the periodontal parameter prediction models described herein are configured to account for patient-specific characteristics when determining periodontal parameters from scan data (also referred to herein as “normalization”). For example, the baseline visual appearance of the gingiva (e.g., color, shape) may vary from patient to patient, such that the absolute color values of the gingival tissue may not be an accurate indicator of inflammation, periodontal disease, etc. As another example, the anatomy of the bone (e.g., bone height) and/or other underlying tissues may affect the appearance of the gingiva. In a further example, previous measurements of periodontal parameters for the patient (e.g., periodontal pocket depth values obtained through probing) may be helpful for predicting current periodontal parameters. Models that consider such patient-specific characteristics may provide more accurate and reliable predictions compared to models that do not.

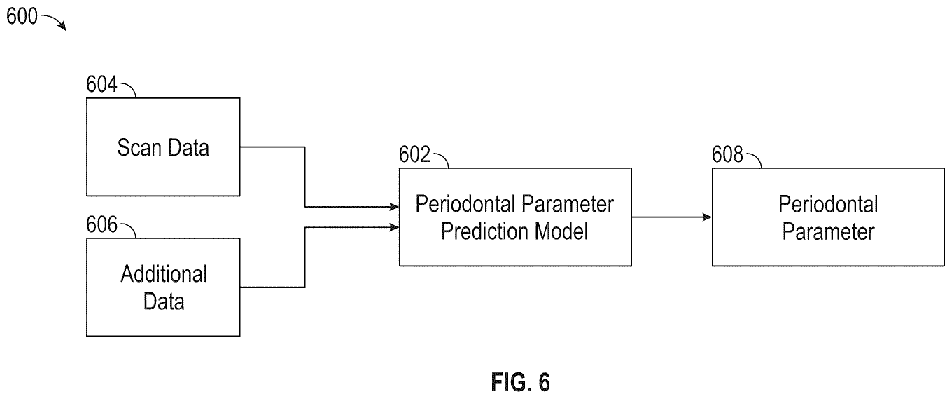

FIG. 6 is a schematic block diagram illustrating a workflow 600 for evaluating a patient's intraoral health using a periodontal parameter prediction model 602, in accordance with embodiments of the present technology. The workflow 600 can be implemented using any of the systems and devices described herein. In some embodiments, some or all of the processes of the workflow 600 are implemented as computer-readable instructions (e.g., program code) that are configured to be executed by one or more processors of a computing device (e.g., a client device, a server device, or suitable combinations thereof).

The periodontal parameter prediction model 602 is a machine learning model that is trained on scan data 604 and additional data 606 to predict a periodontal parameter 608 for one or more of a patient's teeth. The scan data 604 and the periodontal parameter 608 can be identical or generally similar to the scan data 204 and periodontal parameter 208 discussed with reference to FIG. 2. For example, the scan data 604 can include surface topography data, color image data, NIR image data, and/or other data of the dental arch that may be obtained via intraoral scanning. The periodontal parameter 608 can be a quantitative or qualitive indication of the condition of one or more intraoral tissues proximate to and/or supporting a tooth, such as a periodontal pocket depth value, GI, MGI, etc.

The additional data 606 can be any data that is different from the scan data 604, and that provides an indication of patient-specific characteristics that may be relevant to the prediction of the periodontal parameter 608. In some embodiments, the additional data 606 includes data obtained via a different imaging modality than the scan data (e.g., non-scan data), such as x-ray data (e.g., bitewing x-ray data, panoramic x-ray data, cephalometric x-ray data, computed tomography (CT) data, cone-beam computed tomography (CBCT) data, fluoroscopy data), magnetic resonance imaging (MRI) data, photographs, videos, etc. The additional data 606 may alternatively or additionally include data that is determined based on scan data obtained at a different time than the scan data 604 (e.g., previous scan data obtained at an earlier time point). The additional data 606 may alternatively or additionally include data that is determined based on scan data (e.g., which may or may not be the same as the scan data 604), but that is presented in a different format than the scan data (e.g., values, metrics, statistics, and/or other information that is derived from scan data). The additional data 606 may alternatively or additionally include data that is obtained from a different source than the scan data 604, such as data from dental records, electronic health records, user input, etc.

For example, the additional data 606 can include any of the following: demographic information (e.g., age, gender, ethnicity), health condition data (e.g., height, weight, body mass index, blood pressure, diabetes status, smoking status, medications), previous periodontal parameter data (e.g., previously measured periodontal pocket depth values for some or all of the teeth that are obtained, for example, from a periodontal chart or other dental record), palate color data, other imaging modality data (e.g., x-ray data), bone height and/or bone loss data, gum recession data, inflammation data, tooth mobility data, and/or occlusion data. In some embodiments, bone height and/or bone loss can be evaluated based on CBCT data and/or other types of x-ray data. As another example, tooth mobility can be evaluated by obtaining scan data of the patient's dentition when the patient is biting down (“bite scan”) and when the patient's jaws are at rest with substantially no bite forces (“resting scan”). The bite scan and resting scan can be compared to each other to identify differences in tooth position between the two scans, which may be indicative of tooth mobility (e.g., mobile teeth may exhibit positional changes on the order of hundreds of microns when biting forces are applied). Alternatively or in combination, tooth mobility may be evaluated based on manual examination by a clinician; the evaluation results may be provided as part of a dental record or via user input.

The periodontal parameter prediction model 602 may be generally similar to the periodontal parameter prediction model 202 of FIG. 2, except that the periodontal parameter prediction model 602 uses both the scan data 604 and the additional data 606 to generate a prediction of the periodontal parameter 608. In some embodiments, the periodontal parameter prediction model 602 uses the additional data 606 to account for one or more patient-specific characteristics when predicting the periodontal parameter 608, e.g., the additional data 606 is used to “normalize” the output of the periodontal parameter prediction model 602 to the characteristics of the particular patient. The training of the periodontal parameter prediction model 602 can be generally similar to the training workflow illustrated in FIG. 2, except that the training data set also includes additional data representing patient-specific characteristics, in addition to the scan data and the periodontal parameter measurements. Accordingly, the periodontal parameter prediction model 602 can learn how the patient-specific characteristics represented by the additional data correlate to the periodontal parameter measurements.

In some embodiments, the scan data 604 and the additional data 606 are combined (e.g., concatenated) into a single input data set that is provided to the periodontal parameter prediction model 602. For instance, in embodiments where the periodontal parameter prediction model 602 is a CNN (or other machine learning model that operates on array-based input data), the scan data 604 can be processed to generate 2D image data for each gingival region for which a periodontal parameter prediction is to be made, e.g., as described with respect to the method 400 of FIG. 4. The 2D image data may be represented as an array (e.g., a tensor), and the additional data 606 can be concatenated to the array, e.g., as an additional layer in the array, as additional entries in an existing layer in the array (e.g., additional rows, columns), etc. For example, if the 2D image data is represented as an array of 2048 image features, and the additional data 606 includes the patient's age and whether the patient has diabetes, the input data set can be an array of 2050 features: the 2048 image features concatenated to a single feature for the age and a single feature for the diabetes status. As another example, if the 2D image data is represented as an array of 2048 image features, and the additional data 606 includes x-ray data represented as an array of 2048 x-ray features, the input data set can be an array of 4096 features: the 2048 image features concatenated to the 2048 x-ray features. This approach allows a single machine learning model (e.g., a single CNN) to be used to generate predictions based on scan data 604 and additional data 606 concurrently.

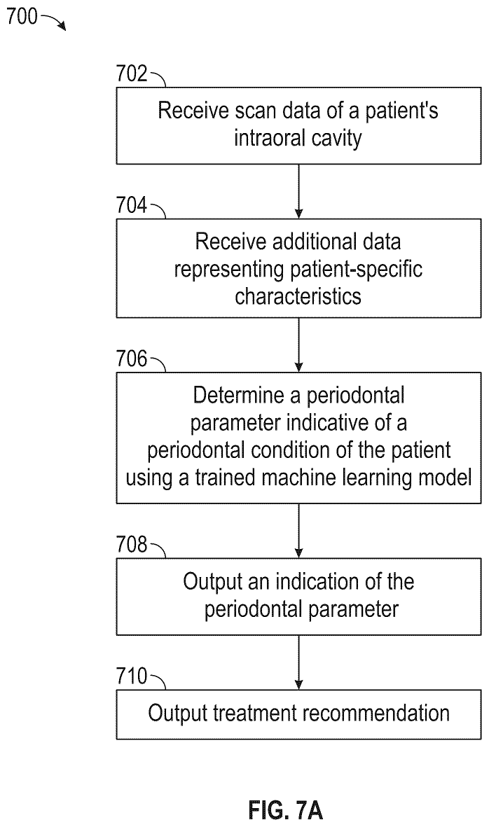

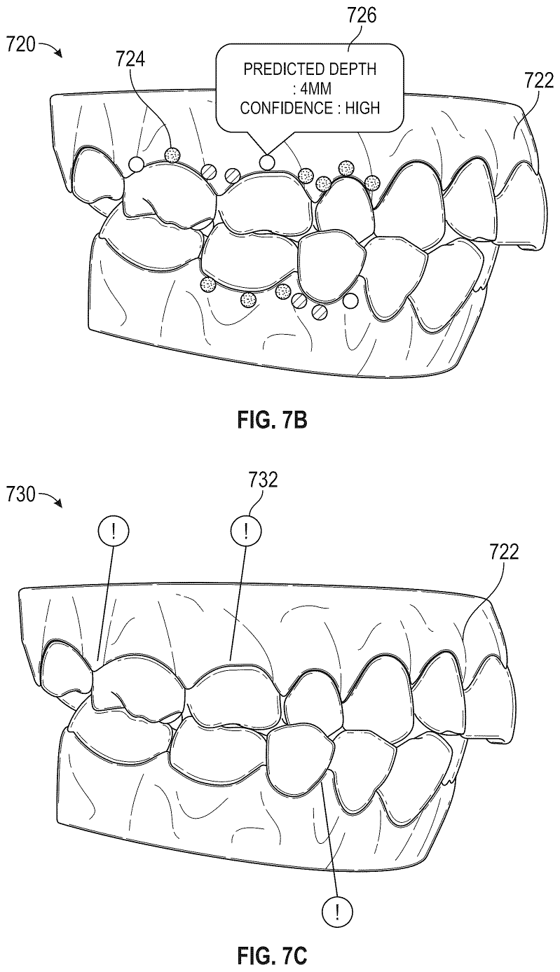

FIG. 7A is a flow diagram illustrating a method 700 for evaluating a patient's intraoral health, in accordance with embodiments of the present technology. The method 700 can be implemented using any of the systems and devices described herein. In some embodiments, some or all of the processes of the method 700 are implemented as computer-readable instructions (e.g., program code) that are configured to be executed by one or more processors of a computing device (e.g., a client device, a server device, or suitable combinations thereof). The method 700 may be combined with any of the other methods described herein, such as the method 400 of FIG. 4.

The method 700 can begin at block 702 with receiving scan data of a patient's intraoral cavity. The scan data can depict one or more intraoral structures of interest, such as the teeth, gingiva, palate, tongue, cheeks, etc. The scan data can include surface topography data, color image data, NIR image data, and/or any other data obtained via intraoral scanning, e.g., as discussed above with respect to FIG. 2. The scan data may be provided in any suitable format, such as a 3D digital representation (e.g., point cloud, height/depth map, surface model, mesh model), a 2D digital representation (e.g., 2D images, continuous texture map), or a combination thereof.

At block 704, the method 700 can include receiving additional data representing one or more patient-specific characteristics of the patient. The additional data can be any data that is different from the scan data, and that provides an indication of patient-specific characteristics that may be relevant to the patient's intraoral health. The additional data may include data obtained via a different imaging modality than the scan data (e.g., non-scan data such as x-ray data, MRI data, photographs, videos, etc.), data that is determined based on scan data obtained at a different time than the scan data of block 702 (e.g., previous scan data), data that is determined based on scan data but that is presented in a different format than the scan data (e.g., values, metrics, statistics, etc., derived from scan data), and/or data than is obtained from a different source than the scan data of block 702 (e.g., data from dental records, electronic health records, user input, etc.). For example, the additional data can include any of the following: demographic information (e.g., age, gender, ethnicity), health condition data (e.g., height, weight, body mass index, blood pressure, diabetes status, smoking status, medications), previous periodontal parameter data (e.g., previously measured periodontal pocket depth values for some or all of the teeth that are obtained, for example, from a periodontal chart or other dental record), palate color data, other imaging modality data (e.g., x-ray data), bone height and/or bone loss data, gum recession data, inflammation data, tooth mobility data, and/or occlusion data. The additional data can be provided in any suitable format, such as 3D digital representations (e.g., 3D models), 2D digital representations (e.g., images), graphs, tables, charts, numerical values, qualitative descriptors, etc. The additional data can be received from various data sources, such as electronic health records (e.g., periodontal charts or other dental records), databases, imaging systems, etc.

At block 706, the method 700 can continue with determining a periodontal parameter indicative of a periodontal condition of the patient using a trained machine learning model. The periodontal parameter can be a quantitative or qualitive indication of the condition of one or more intraoral tissues proximate to and/or supporting a tooth, e.g., as discussed above with respect to FIG. 2. For example, the periodontal parameter can be a periodontal pocket depth value, GI, MGI, etc.

In some embodiments, the periodontal parameter is predicted using a machine learning model (e.g., the periodontal parameter prediction model 602 of FIG. 6) that is trained on scan data and periodontal parameter measurements from a plurality of patients. The machine learning model can be configured to use the additional data to account for the one or more patient-specific characteristics when predicting the periodontal parameter, e.g., as discussed above with respect to FIG. 6. For example, an input data set can be generated by combining the scan data and the additional data, the input data set can be input into the machine learning model, and the output of the machine learning model can be the periodontal parameter.

The periodontal parameter may be predicted for one or more regions of interest in the patient's intraoral cavity, such as a gingival region proximate to a tooth (e.g., a lingual region, a buccal region, a mesial region, a distal region, or a combination thereof). In some embodiments, a periodontal parameter is determined for each of the patient's teeth, or for each of a subset of the patient's teeth. The periodontal parameter may be a local parameter (e.g., a parameter for a specific gingival region or a specific tooth) or may be a global parameter (e.g., a parameter for a group of teeth such as quadrant or sextant of the dental arch, or for an entire dental arch). A global parameter may be obtained by combining (e.g., summing) and/or calculating statistics (e.g., minimum, maximum, mean) from a plurality of local parameters. Optionally, multiple periodontal parameters may be determined, some of which are local parameters and some of which are global parameters.

Each predicted periodontal parameter may optionally be associated with a confidence score, which may be a numerical value (e.g., a percentage or probability value), a quantitative assessment (e.g., “high confidence,” “low confidence”), or a combination thereof that represents the estimated degree of accuracy of the prediction. The confidence score may be produced by the same algorithm that predicts the periodontal parameter (e.g., the periodontal parameter prediction model 602 of FIG. 6).

At block 708, the method 700 can include outputting an indication of the periodontal parameter. The indication of the periodontal parameter can be presented in many different formats, such as numerically (e.g., depth values, index values, scores), textually (e.g., categorizations, descriptive assessments, notifications), and/or graphically. In some embodiments, one or more indicators (e.g., markers, icons, coloring, shading, labels) are overlaid onto or otherwise displayed together with a digital representation of the teeth (e.g., a 3D digital model, a 2D image), where the indicators show the locations at which the periodontal parameters were determined and provide a visual representation of the values of the periodontal parameters. This approach can be advantageous, for example, to assist a user (e.g., the patient and/or the clinician) in visually identifying regions of the intraoral cavity where periodontal issues may be present.

FIG. 7B illustrates a user interface 720 for displaying predicted periodontal parameters of a patient, in accordance with embodiments of the present technology. The user interface 720 may be output by a display (e.g., a monitor, touchscreen) of a computing device (e.g., a mobile device (such as a smartphone), tablet, laptop, workstation). The user interface 720 can display a digital model 722 of one or both dental arches of the patient. The digital model 722 can be a 3D model generated from intraoral scan data, as discussed elsewhere herein. In the illustrated embodiment, a set of indicators 724 (e.g., markers) representing periodontal parameters (e.g., periodontal pocket depth values) are overlaid onto the digital model 722 at the locations where predictions were made, with the color of the indicator 724 corresponding to the value for the parameter (e.g., green=normal pocket depth values, yellow=intermediate pocket depth values, red=deep pocket depth values). Alternatively or in combination, the parameter values may be represented by different indicator shapes, sizes, etc. Moreover, the indicators 724 can alternatively be displayed as a shaded or colored region, such as an overlay (e.g., a heatmap), rather than discrete markers as shown in FIG. 7B. The overlay may be applied to a portion of or the entirety of the digital model 722, for example, via texture mapping. Optionally, an individual indicator 724 may be selected (e.g., via clicking, hovering) to display a tooltip 726 (or other graphical element) that provides additional relevant information, such as the predicted value, a confidence score for the prediction, a previously measured or predicted value, recommended action items (e.g., “perform physical probing”), etc.

FIG. 7C illustrates another user interface 730 for displaying predicted periodontal parameters of a patient, in accordance with embodiments of the present technology. The user interface 730 may be generally similar to the user interface 720 of FIG. 7B, except that indicators 724 are only displayed for locations where abnormal values are predicted (e.g., deep pocket depth values) and/or where an action is recommended (e.g., physical probing). Selection of an individual indicator 732 may result in display of additional relevant information, e.g., similar to the tooltip 726 of FIG. 7B.

Referring again to FIG. 7A, in some embodiments, the process of block 708 includes outputting a notification if the periodontal parameter is determined to be abnormal. An abnormal periodontal parameter may be detected by comparing the value of the predicted periodontal parameter to a threshold value, which may be a generic threshold value or a patient-specific threshold value (e.g., a threshold that accounts for patient-specific characteristics such as baseline periodontal parameter values). The notification can be provided in any suitable format, such as text, graphics, audible alerts, haptic alerts, etc.

At block 710, the method 700 can optionally include outputting a treatment recommendation, based on the determined periodontal parameter. The treatment recommendation can include one or more actions to be performed by the clinician and/or patient to address a disease or condition that may be present, as indicated by the value of the periodontal parameter. For instance, an abnormal periodontal parameter value at a particular location (e.g., a value falling outside of a normal range and/or within a range associated with disease) may produce a recommendation that the clinician perform additional diagnostic procedures on that location (e.g., visual inspection, physical probing, take x-rays) to confirm that that the abnormal value is accurate and/or to identify any pathology present at that location.

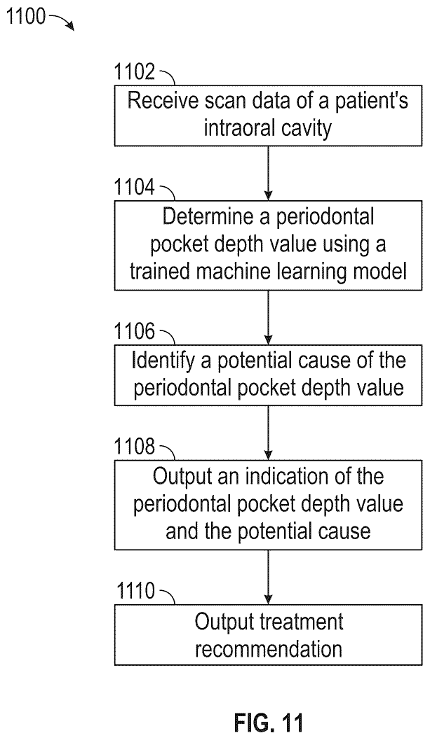

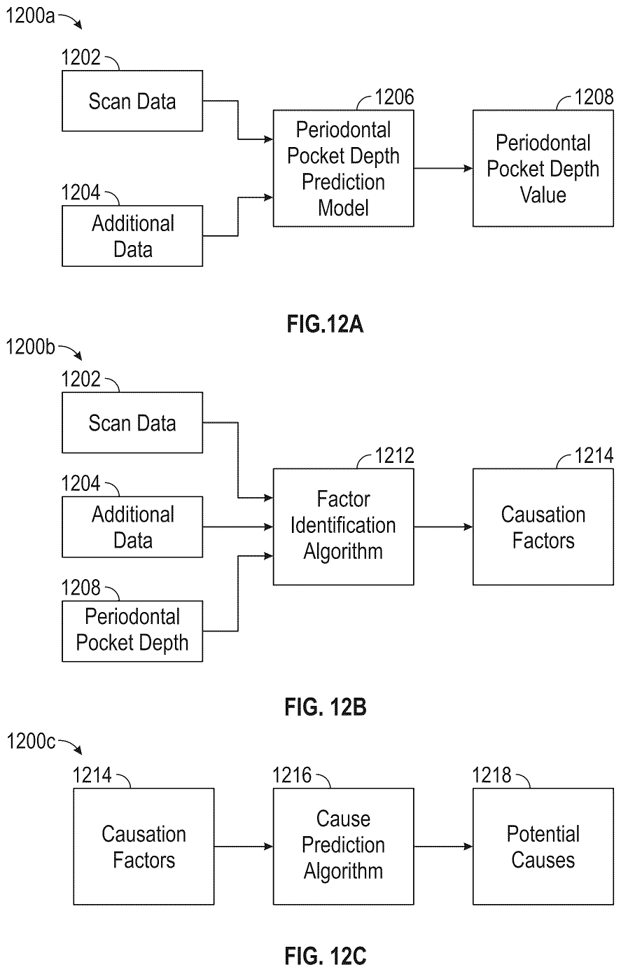

As another example, if the cause of an abnormal periodontal parameter value is ascertainable, the treatment recommendation can include an action to address the cause. For instance, deep periodontal pockets may be caused by poor oral hygiene (which may be addressed by improving brushing and flossing habits) and/or by bruxism (which may be addressed by wearing a night guard). Additional details and examples of methods for predicting causes of abnormal periodontal parameter values are discussed below in connection with FIGS. 11-12C.

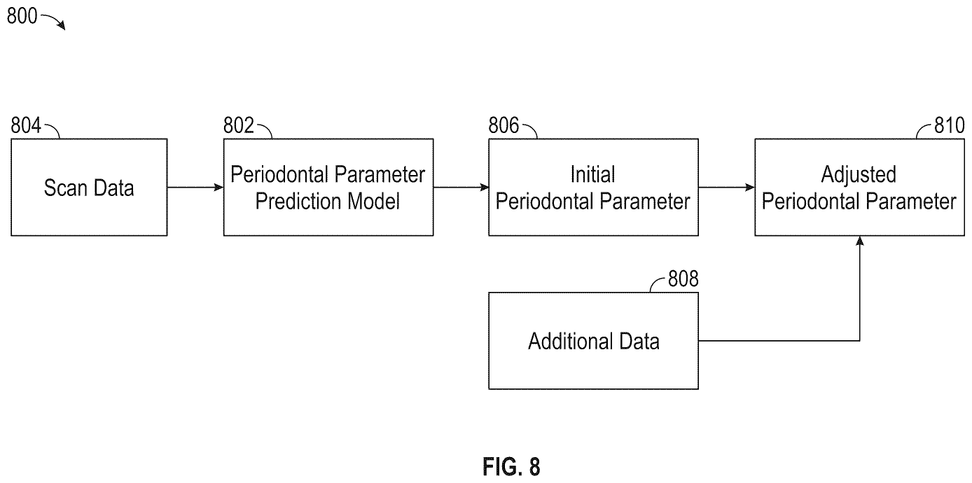

FIG. 8 is a schematic block diagram illustrating a workflow 800 for evaluating a patient's intraoral health using a periodontal parameter prediction model 802, in accordance with embodiments of the present technology. The workflow 800 can be implemented using any of the systems and devices described herein. In some embodiments, some or all of the processes of the workflow 800 are implemented as computer-readable instructions (e.g., program code) that are configured to be executed by one or more processors of a computing device (e.g., a client device, a server device, or suitable combinations thereof).

The periodontal parameter prediction model 802 is a machine learning model (e.g., a CNN or other deep learning neural network) that is trained on scan data 804 to predict an initial periodontal parameter 806 for one or more of a patient's teeth. The scan data 804 and the initial periodontal parameter 806 can be identical or generally similar to the scan data 204 and periodontal parameter 208 discussed with reference to FIG. 2. For example, the scan data 804 can include surface topography data, color image data, NIR image data, and/or other data of the dental arch that may be obtained via intraoral scanning. The initial periodontal parameter 806 can be a quantitative or qualitive indication of the condition of one or more intraoral tissues proximate to and/or supporting a tooth, such as a periodontal pocket depth value, GI, MGI, etc.

The periodontal parameter prediction model 802 can be identical or generally similar to the periodontal parameter prediction model 202 of FIG. 2. In some embodiments, the periodontal parameter prediction model 802 is trained to determine the initial periodontal parameter 806 using the scan data 804 only, without considering additional data representing patient-specific characteristics. Accordingly, initial periodontal parameter 806 produced by the periodontal parameter prediction model 802 may be a “generic” prediction, in that it does not account for the effects of patient-specific characteristics (e.g., color and/or shape of the gingiva, previous periodontal parameter measurements) on the periodontal parameter. The training of the periodontal parameter prediction model 802 may be identical or generally similar to the training workflow of FIG. 2.

In some embodiments, if additional data 808 representing patient-specific characteristics is available, the initial periodontal parameter 806 produced by the periodontal parameter prediction model 802 can subsequently be adjusted based on the additional data 808 to generate an adjusted periodontal parameter 810, where the adjustment is made to account for one or more patient-specific characteristics. The additional data 808 can be identical or generally similar to the additional data 606 discussed with reference to FIG. 6. For example, the additional data 808 can be any data that is different from the scan data 804, and that provides an indication of patient-specific characteristics that may be relevant to the periodontal parameter prediction, such as data obtained via a different imaging modality than the scan data 804 (e.g., non-scan data such as x-ray data, MRI data, photographs, videos, etc.), data that is determined based on scan data obtained at a different time than the scan data 804 (e.g., previous scan data), data that is determined based on scan data but that is presented in a different format than the scan data 804 (e.g., values, metrics, statistics, etc., derived from scan data), and/or data than is obtained from a different source than the scan data 804 (e.g., data from dental records, electronic health records, user input, etc.). For example, the additional data 808 can include demographic information, health condition data, previous periodontal parameter data, palate color data, other imaging modality data, bone height and/or bone loss data, gum recession data, inflammation data, tooth mobility data, and/or occlusion data.

The adjusted periodontal parameter 810 can be generated based on the initial periodontal parameter 806 and the additional data 808 in various ways. For example, the additional data 808 may be used to correct the initial periodontal parameter 806, e.g., based on a previous prediction error of the periodontal parameter prediction model 802. For example, in embodiments where the additional data 808 includes previous measurements of periodontal parameters and previous predictions of the periodontal parameters produced using the periodontal parameter prediction model 802, the previous measurements can be compared to the previous predictions to determine an error in the predictions produced by the periodontal parameter prediction model 802 (e.g., the difference between the predicted value and the measured value). Prediction errors may be determined for an individual gingival region, for an individual tooth, or for groups of teeth (e.g., sextants, quadrants, or an entire dental arch). The error can then be used to correct the initial periodontal parameter 806, e.g., by adjusting the value of the initial periodontal parameter 806 to compensate for the error. For example, if previous periodontal pocket depth values predicted by the periodontal parameter prediction model 802 were determined to be on average 1 mm less than the actual measurements for posterior teeth and 2 mm less on the measurements for anterior teeth, the currently predicted periodontal pocket depth values can be adjusted up by 1 mm for the posterior teeth and by 2 mm for the anterior teeth. As another example, if all of the previous periodontal pocket depth values were too high by 1 mm, a global correction of −1 mm can be applied to all of the currently predicted periodontal pocket depth values.

Alternatively or in combination, the additional data 808 may be analyzed (e.g., using a software algorithm such as a machine learning model and/or a rule-based algorithm) to determine a correction that should be applied to the initial periodontal parameter 806. For instance, information regarding the baseline color of the patient's soft tissue (e.g., palate color data) may be used to determine a correction that adjusts the initial periodontal parameter 806 to account for patient-specific color variations. A single correction may be applied globally to all periodontal parameters, or different corrections may be applied to different periodontal parameters.

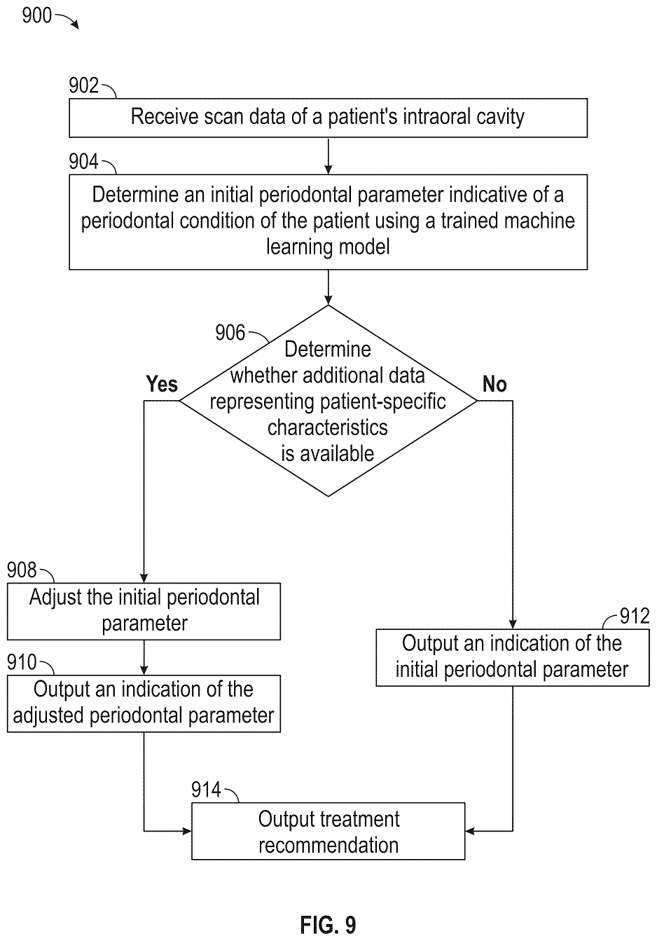

FIG. 9 is a flow diagram illustrating a method 900 for evaluating a patient's intraoral health, in accordance with embodiments of the present technology. The method 900 can be implemented using any of the systems and devices described herein. In some embodiments, some or all of the processes of the method 900 are implemented as computer-readable instructions (e.g., program code) that are configured to be executed by one or more processors of a computing device (e.g., a client device, a server device, or suitable combinations thereof). The method 900 may be combined with any of the other methods described herein, such as the method 400 of FIG. 4.

The method 900 can begin at block 902 with receiving scan data of a patient's intraoral cavity. The scan data can depict one or more intraoral structures of interest, such as the teeth, gingiva, palate, tongue, cheeks, etc. The scan data can include surface topography data, color image data, NIR image data, and/or any other data obtained via intraoral scanning, e.g., as discussed above with respect to FIG. 2. The scan data may be provided in any suitable format, such as a 3D digital representation (e.g., point cloud, height/depth map, surface model, mesh model), a 2D digital representation (e.g., 2D images, continuous texture map), or a combination thereof.

At block 904, the method 900 can continue with determining an initial periodontal parameter indicative of a periodontal condition of the patient using a trained machine learning model. The initial periodontal parameter can be a quantitative or qualitive indication of the condition of one or more intraoral tissues proximate to and/or supporting a tooth, e.g., as discussed above with respect to FIG. 2. For example, the periodontal parameter can be a periodontal pocket depth value, GI, MGI, etc. The initial periodontal parameter may be predicted for one or more regions of interest in the patient's intraoral cavity, such as a gingival region proximate to a tooth (e.g., a lingual region, a buccal region, a mesial region, a distal region, or a combination thereof). In some embodiments, an initial periodontal parameter is determined for each of the patient's teeth, or for each of a subset of the patient's teeth. The initial periodontal parameter may be a local parameter (e.g., a parameter for a specific gingival region or a specific tooth) or may be a global parameter (e.g., a parameter for a group of teeth or for an entire dental arch). A global parameter may be obtained by combining (e.g., summing) and/or calculating statistics (e.g., minimum, maximum, mean) from a plurality of local parameters. Optionally, multiple initial periodontal parameters may be determined, some of which are local parameters and some of which are global parameters.

The initial periodontal parameter can be predicted using a machine learning model (e.g., the periodontal parameter prediction model 802 of FIG. 8) that is trained on scan data and periodontal parameter measurements from a plurality of patients. In some embodiments, the machine learning model is trained to predict the initial periodontal parameter using the scan data only, without considering additional data representing patient-specific characteristics.

At block 906, the method 900 can include determining whether additional data representing one or more patient-specific characteristics of the patient is available. The additional data can be any data that is different from the scan data, and that provides an indication of patient-specific characteristics that may be relevant to the patient's intraoral health. The additional data may include data obtained via a different imaging modality than the scan data (e.g., non-scan data such as x-ray data, MRI data, photographs, videos, etc.), data that is determined based on scan data obtained at a different time than the scan data of block 902 (e.g., previous scan data), data that is determined based on scan data but that is presented in a different format than the scan data (e.g., values, metrics, statistics, etc., derived from scan data), and/or data than is obtained from a different source than the scan data of block 902 (e.g., data from dental records, electronic health records, user input, etc.). For example, the additional data can include any of the following: demographic information (e.g., age, gender, ethnicity), health condition data (e.g., height, weight, body mass index, blood pressure, diabetes status, smoking status, medications), previous periodontal parameter data (e.g., previously measured periodontal pocket depth values for some or all of the teeth that are obtained, for example, from a periodontal chart or other dental record), palate color data, other imaging modality data (e.g., x-ray data), bone height and/or bone loss data, gum recession data, inflammation data, tooth mobility data, and/or occlusion data. The additional data can be provided in any suitable format, such as 3D digital representations (e.g., 3D models), 2D digital representations (e.g., images), graphs, tables, charts, numerical values, qualitative descriptors, etc. The additional data can be received from various data sources, such as electronic health records, databases, imaging systems, etc.

If the additional data is available, the method 900 can proceed to block 908 with adjusting the initial periodontal parameter based on the additional data. For example, the adjustment can include applying a correction to the periodontal parameter that accounts for one or more patient-specific characteristics. In some embodiments, the correction is based on previous scan data and previous parameter data for the patient. For instance, a predicted periodontal parameter can be determined using the machine learning model and the previous scan data, and the predicted periodontal parameter can be compared to an actual periodontal parameter from the previous periodontal parameter data. The initial periodontal parameter can then be adjusted based on the comparison, e.g., by identifying an error between the predicted periodontal parameter from the previous scan data and the actual periodontal parameter from the previous periodontal parameter data, and determining a correction value to compensate for the error.

Subsequently, the method 900 can continue to block 910 with outputting an indication of the adjusted periodontal parameter. The indication of the periodontal parameter can be presented in many different formats, such as numerically (e.g., depth values, index values, scores), textually (e.g., categorizations, descriptive assessments, notifications), and/or graphically. In some embodiments, one or more indicators (e.g., markers, icons, coloring, shading, labels) are overlaid onto or otherwise displayed together with a digital representation of the teeth (e.g., a 3D digital model, a 2D image), where the indicators show the locations at which the periodontal parameters were determined and provide visual representation of the values of the periodontal parameters (e.g., as illustrated in the embodiments of FIGS. 7B and 7C). If the adjusted periodontal parameter has an abnormal value (e.g., based on a comparison of the adjusted periodontal parameter value to a threshold value), the indication may also include a notification alerting the user to the abnormal value. Optionally, the indication may provide a notification that an adjustment was made to the periodontal parameter based on additional data and may optionally provide relevant information regarding the adjustment (e.g., the data used as a basis for the adjustment, the amount of the adjustment).

If the additional data is not available, the method 900 can instead proceed to block 912 with outputting an indication of the initial periodontal parameter. The process of block 912 can be generally similar to the process of block 910, except that the indication of the initial periodontal parameter may optionally indicate that no additional data was available for making patient-specific adjustments.

At block 914, the method 900 can optionally include outputting a treatment recommendation, based on the adjusted periodontal parameter of block 910 or the initial periodontal parameter of block 912. The treatment recommendation can include one or more actions to be performed by the clinician and/or patient to address a disease or condition that may be present, as indicated by the value of the periodontal parameter. For instance, an abnormal periodontal parameter value at a particular location (e.g., a value falling outside of a normal range and/or within a range associated with disease) may produce a recommendation that the clinician perform additional diagnostic procedures on that location (e.g., visual inspection, physical probing, take x-rays) to confirm that that the abnormal value is accurate and/or to identify any pathology present at that location. As another example, if the cause of an abnormal periodontal parameter value is ascertainable, the treatment recommendation can include an action to address the cause. Additional details and examples of methods for predicting causes of abnormal periodontal parameter values are discussed below in connection with FIGS. 11-12C.

In some embodiments, normalization of periodontal parameter predictions is achieved by developing periodontal parameter prediction models that are trained or otherwise configured for a particular patient, e.g., the model parameters (e.g., neural network weights) for the model are customized to the patient. Such models may be produced by training a generic model on scan data and/or additional (e.g., non-scan) data for the particular patient, thereby resulting in a patient-specific model that has been trained to learn correlations between scan data and/or additional data and periodontal parameters for the particular patient. Alternatively or in combination, such models may be produced by training a generic model based on errors between predicted and actual values of periodontal parameters for a particular patient, thereby resulting in a patient-specific model that has been trained to compensate for such errors in determining the predicted periodontal parameters for the particular patient.

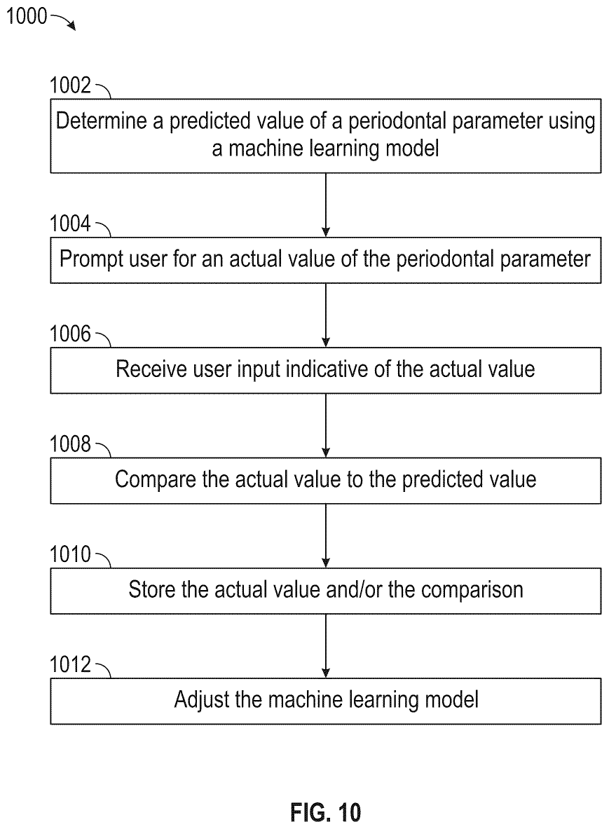

FIG. 10 is a flow diagram illustrating a method 1000 for generating a patient-specific periodontal parameter prediction model, in accordance with embodiments of the present technology. The method 1000 can be implemented using any of the systems and devices described herein. In some embodiments, some or all of the processes of the method 1000 are implemented as computer-readable instructions (e.g., program code) that are configured to be executed by one or more processors of a computing device (e.g., a client device, a server device, or suitable combinations thereof). The method 1000 may be combined with any of the other methods described herein, such as the method 400 of FIG. 4, the method 700 of FIG. 7A, and/or the method 900 of FIG. 9.

The method 1000 can begin at block 1002 with determining a predicted value of a periodontal parameter using a machine learning model. The machine learning model can be trained to predict the periodontal parameter value based on scan data and, optionally, additional data representing one or more patient-specific characteristics. For example, the machine learning model can be the periodontal parameter prediction model 202 of FIG. 2, the periodontal parameter prediction model 602 of FIG. 6, or the periodontal parameter prediction model 802 of FIG. 8.

At block 1004, the method 1000 can continue with prompting a user (e.g., a clinician) for an actual value of the periodontal parameter. For example, a prompt can be displayed on a user interface of a display (e.g., a monitor, touchscreen) of a computing device (e.g., a mobile device (such as a smartphone), tablet, laptop, workstation). The prompt can include instructions for the user to obtain the actual value, e.g., via physical examination (e.g., probing) of the patient's intraoral cavity, by looking up the actual value from a periodontal chart or other dental record, etc. The prompt can indicate the location where the actual value should be obtained, which may correspond to the particular gingival region and/or tooth for which the predicted value of the periodontal parameter was determined. Optionally, the prompt can display the predicted value of the periodontal parameter, e.g., as a reference or baseline for the user in obtaining the actual value of the periodontal parameter.

At block 1006, the method 1000 can include receiving user input indicative of the actual value of the periodontal parameter. The user input can be provided using any suitable input device, such as a mouse, keyboard, touchscreen, etc.

At block 1008, the method 1000 can compare the actual value of the periodontal parameter to the predicted value of the periodontal parameter, e.g., to determine whether the actual value is greater than, less than, or substantially equal to the predicted value. The comparison can be performed to identify an error (e.g., difference) between the actual value and the predicted value.

At block 1010, the method 1000 can optionally include storing the actual value and/or the comparison. The actual value and/or comparison can be stored in any suitable data store, such as a database, dental record, training dataset, etc. The stored actual value and/or comparison may subsequently be retrieved and displayed to a user when generating subsequent predictions of the periodontal parameter for the patient using the machine learning model, e.g., to provide a reference for evaluating the accuracy of the prediction and/or to allow for correction of future predicted values (e.g., as discussed with respect to the embodiments of FIGS. 8 and 9).

At block 1012, the method 1000 can include adjusting the machine learning model based on the actual value and/or the comparison. The adjustment can include retraining the machine learning model using the actual value and/or comparison as part of the training data set. For example, the retraining can result in adjustments to the parameters of the machine learning model to correct the error between the actual and predicted values for the periodontal parameter. In some embodiments, the machine learning model used in block 1002 is a generic model, and the adjustment in block 1012 results in a patient-specific model that accounts for patient-specific characteristics when predicting the value of a periodontal parameter. In some embodiments, the machine learning model used in block 1002 is a patient-specific model, and the adjustment in block 1012 further refines the patient-specific model to provide more accurate predictions for the particular patient. The adjusted model can thus be used to make subsequent predictions of the patient's periodontal parameters with improved accuracy and reliability, compared to a generic model.

In some embodiments, a method for assessing a patient's intraoral health includes determining whether an adjusted machine learning model that has been trained on patient-specific data for the patient is available (e.g., an adjusted model produced according to the method 1000 of FIG. 10). If the adjusted machine learning model is available, the adjusted machine learning model is used to determine a periodontal parameter for the patient. If the adjusted machine learning model is not available, a generic machine learning model that has not been trained on patient-specific data for the patient is used determine the periodontal parameter. The generic machine learning model may subsequently be adjusted to produce an adjusted machine learning model (e.g., according to the method 1000 of FIG. 10).

Although some embodiments of the normalization techniques described herein use non-scan data in combination with scan data to account for patient-specific characteristics, in other embodiments, the “additional data” may include data that is extracted from or otherwise generated based on the scan data. For example, continuous texture maps generated from color image data (e.g., as shown in FIG. 3C) may show the baseline color of the patient's soft tissues (e.g., palate color) and thus may be used to generate periodontal parameter predictions that account for patient-specific variations in soft tissue color. In such embodiments, a periodontal parameter prediction model may use the continuous texture map (or a selected region thereof, such a cropped region depicting the palate only) as “additional data” in determining a periodontal parameter for the patient, e.g., as previously discussed with respect to FIGS. 6-9.

### C. Causation Analysis

In some embodiments, the present technology provides methods for identifying a potential cause of an abnormal intraoral condition, such as an abnormal periodontal pocket depth. Identification of the potential cause may be beneficial, for example, for assisting the clinician and/or user in determining treatment recommendations to address the abnormal periodontal parameter value, such as improving oral hygiene, treating bruxism via a night guard or other protective device, etc. Although the following embodiments of FIGS. 11-12C describe identification of potential causes of abnormal periodontal pocket depth values, the present technology may also be used to identify the potential causes of other abnormal periodontal parameters (e.g. MGI, GI) and/or other parameters relevant to intraoral health.

FIG. 11 is a flow diagram illustrating a method 1100 for evaluating a patient's intraoral health, in accordance with embodiments of the present technology. The method 1100 can be implemented using any of the systems and devices described herein. In some embodiments, some or all of the processes of the method 1100 are implemented as computer-readable instructions (e.g., program code) that are configured to be executed by one or more processors of a computing device (e.g., a client device, a server device, or suitable combinations thereof). The method 1100 may be combined with any of the other methods described herein, such as any of the methods described in Sections I.A and I.B above.

The method 1100 can begin at block 1102 with receiving scan data of a patient's intraoral cavity. The scan data can depict one or more intraoral structures of interest, such as the teeth, gingiva, palate, tongue, cheeks, etc. The scan data can include surface topography data, color image data, NIR image data, and/or any other data obtained via intraoral scanning, e.g., as discussed above with respect to FIG. 2. The scan data may be provided in any suitable format, such as a 3D digital representation (e.g., point cloud, height/depth map, surface model, mesh model), a 2D digital representation (e.g., 2D images, continuous texture map), or a combination thereof.

At block 1104, the method 1100 can include determining a periodontal pocket depth value for the patient using a trained machine learning model, based on the scan data. The machine learning model can be or include any of the periodontal parameter prediction models described in Sections I.A and I.B above (e.g., the periodontal parameter prediction model 202 of FIG. 2, the periodontal parameter prediction model 602 of FIG. 6, the periodontal parameter prediction model 802 of FIG. 8).

FIG. 12A is a schematic block diagram illustrating a workflow 1200a for determining a periodontal pocket depth value, in accordance with embodiments of the present technology. As shown in FIG. 12A, scan data 1202 and, optionally, additional data 1204 can be provided to a periodontal pocket depth prediction model 1206, and the periodontal pocket depth prediction model 1206 can produce a prediction of a periodontal pocket depth value 1208. The periodontal pocket depth prediction model 1206 can include a machine learning model that is trained on scan data and periodontal parameter measurements from a plurality of patients to predict a periodontal pocket depth value from scan data. Optionally, the periodontal pocket depth prediction model 1206 can be trained to make predictions that account for patient-specific characteristics using the additional data 1204 (e.g., demographic information, health condition data, previous periodontal pocket depth values for some or all of the teeth, palate color data, other imaging modality data, bone height and/or bone loss data, gum recession data, inflammation data, tooth mobility data, occlusion data), or the initial prediction produced by the periodontal pocket depth prediction model 1206 can subsequently be adjusted based on the additional data 1204 to produce a periodontal pocket depth value 1208 that accounts for such patient-specific characteristics. In other embodiments, however, the periodontal pocket depth prediction model 1206 can determine the periodontal pocket depth value 1208 based on the scan data 1202 only.

Referring again to FIG. 11, at block 1106, the method 1100 can include identifying at least one potential cause of the periodontal pocket depth value. A potential cause may be identified if the periodontal pocket depth value is determined to be abnormal, e.g., the value exceeds a threshold value associated with periodontal disease or other pathological condition. The potential cause of an abnormal periodontal pocket depth value may be identified in many different ways. For example, the potential cause can be identified based on the scan data (e.g., surface topography data, color image data, NIR image data), based on one or more determined periodontal pocket depth values (e.g., the magnitudes and/or locations of one or more abnormal periodontal pocket depth values), and/or based on additional data for the patient (e.g., demographic information, health condition data, previous periodontal parameter data, palate color data, other imaging modality data, bone height and/or bone loss data, gum recession data, inflammation data, tooth mobility data, occlusion data).

In some embodiments, the scan data, periodontal pocket depth values, and/or additional data are analyzed to identify one or more causation factors, where the causation factors represent characteristics of the intraoral cavity that are correlated with potential causes of abnormal periodontal pocket depth values. The causation factors may include calculus build up, increased tooth wear, increased occlusal contacts, bite force, frequency of deep periodontal pocket depths, distribution of deep periodontal pocket depths, blood pressure, diabetes status, and smoking status. Examples of causation factors and the corresponding potential causes are provided in Table 1 below.

TABLE 1
Causation Factors and Potential Causes of
Abnormal Periodontal Pocket Depth Values

| Causation Factors | Potential Cause |
|---|---|
| Gingival inflammation | Poor oral hygiene, periodontal disease |
| Calculus build up | Poor oral hygiene, periodontal disease |
| Increased tooth wear | Bruxism |
| Increased occlusal contacts | Bruxism |

In some embodiments, a plurality of periodontal pocket depth values are determined for multiple locations in the upper and/or lower dental arch of a patient. The number and/or distribution of abnormal periodontal pocket depth values can be a causation factor that provides information relevant to identifying the potential cause of the abnormal values, e.g., how many periodontal pocket depth values exceed a threshold value (e.g., for the upper jaw only, the lower jaw only, or across both jaws), a comparison of the number of abnormal values in the upper jaw versus the lower jaw, etc.

In some embodiments, information regarding the condition of intraoral tissues proximate to the location of an abnormal periodontal pocket depth value can be a causation factor that provides insight into the potential cause of the abnormal value. For example, bruxism may contribute to deep periodontal pockets, and may be detected based on increased tooth wear and/or increased occlusal contacts proximate to the location of the deep pocket. Increased tooth wear and/or occlusal contacts may be detected based on scan data (e.g., based on changes in the surface topography of the teeth over time) and/or occlusal data (e.g., an occlusogram showing the bite contacts and/or forces across different teeth).

As another example, poor oral hygiene may cause periodontal disease that leads to deep periodontal pockets, and may be detected based on inflammation and/or calculus build up on teeth proximate to the location of the deep pocket. Inflammation and/or calculus build up may be detected based on scan data (e.g., color data showing increased redness of gingival tissue and/or plaques on the teeth).

In some embodiments, the potential cause is identified using a software algorithm. The software algorithm can be or include a rule-based algorithm, such as a heuristic decision tree. A heuristic decision tree can be built by collecting information regarding the relationships between various causation factors and potential causes. Such information can be obtained from previous patient data, experimental data, scientific literature, public health databases, etc. Alternatively or in combination, the potential cause can be identified using a machine learning model that has been trained on data for a plurality of patients. For example, the training data can include patient data (e.g., scan data, periodontal pocket depth values, additional data) that has been labeled with the corresponding causes, such that the machine learning model can learn the relationship between features in the patient data and the causes.

In some embodiments, the potential cause is identified using at least two software algorithms: a first algorithm that detects one or more causation factors present in the patient data (e.g., scan data, periodontal pocket depth values, additional data), and a second algorithm that identifies one or more potential causes based on the detected causation factors. The first algorithm and the second algorithm can each independently be a rule-based algorithm, a machine learning model, or a combination thereof.

FIG. 12B is a schematic block diagram illustrating a workflow 1200b for identifying causation factors from patient data, in accordance with embodiments of the present technology. In the illustrated embodiment, scan data 1202, additional data 1204, and one or more periodontal pocket depth values 1208 may be provided to a factor identification algorithm 1212 (e.g., a rule-based algorithm, a machine learning model), and the factor identification algorithm 1212 can analyze the data to determine one or more causation factors 1214 that are present. In other embodiments, however, the factor identification algorithm 1212 may use only a subset of the data to determine the causation factors 1214, such as the scan data 1202 only, the additional data 1204 only, the periodontal pocket depth values 1208 only, a combination of the scan data 1202 and the additional data 1204, etc.

FIG. 12C is a schematic block diagram illustrating a workflow 1200c for determining potential causes of abnormal periodontal pocket depth values based on causation factors, in accordance with embodiments of the present technology. As shown in FIG. 12C, the causation factors 1214 produced by the factor identification algorithm 1212 of FIG. 12B can be provided to a cause prediction algorithm 1216 (e.g., a rule-based algorithm such as a heuristic decision tree, a machine learning model), and the cause prediction algorithm 1216 can determine one or more potential causes 1218 based on the causation factors 1214.

Referring again to FIG. 11, the process of block 1106 can include identifying a single potential cause or multiple potential causes for an abnormal periodontal pocket depth value. Each identified potential cause may optionally be associated with a confidence score, which may be a numerical value (e.g., a percentage or probability value), a quantitative assessment (e.g., “high confidence,” “low confidence”), or a combination thereof that represents the estimated degree of accuracy of the potential cause. The confidence score may be produced by the same algorithm that determines the potential cause (e.g., the cause prediction algorithm 1216 of FIG. 12C). In embodiments where multiple potential causes are identified, the potential causes may be ranked and/or selected based on confidence score. For example, a potential cause may be identified as such only if the associated confidence score is sufficiently high (e.g., above a threshold value). As another example, the potential causes produced by the algorithm may be ranked based on their confidence scores, and only the cause(s) having the highest score(s) may be selected.

At block 1108, the method 1100 can include outputting an indication of the periodontal pocket depth value and the potential cause. The indication of the periodontal pocket depth value can be presented in many different formats, such as numerically, textually, and/or graphically. In some embodiments, one or more indicators are overlaid onto or otherwise displayed together with a digital representation of the teeth, where the indicators show the magnitude and locations at which the periodontal pocket depth values (e.g., as illustrated in the embodiments of FIGS. 7B and 7C).

The potential cause of the periodontal pocket depth value can also be presented in any suitable format, such as numerically, textually, and/or graphically. Each potential cause may be displayed together with the corresponding periodontal pocket depth value, e.g., via information on a tooltip that is displayed upon selection of the periodontal pocket depth value; in a table, graphic, report that is provided to the user with the periodontal pocket depth value; etc. The potential causes may also be displayed together with their associated confidence scores, if available. In embodiments where multiple potential causes are identified for a periodontal pocket depth value, the confidence scores may be used to select a subset of the potential causes for display (e.g., only the potential cause(s) having the highest score(s) are displayed) and/or the potential causes may be displayed in ranked order based on confidence score.

At block 1110, the method 1100 can optionally include outputting a treatment recommendation, based on the potential cause. The treatment recommendation can include one or more actions to be performed by the clinician and/or patient to address a disease or condition that may be present, as indicated by an abnormal periodontal pocket depth value and/or the associated potential cause. Treatment recommendations that may be provided include, for example, performing additional diagnostic procedures to confirm the abnormal periodontal pocket depth value and/or potential cause (e.g., visual inspection, physical probing, x-rays) and/or performing therapeutic interventions (e.g., improving oral hygiene if the potential cause is poor oral hygiene and/or periodontal disease, wearing a night guard if the potential cause is bruxism).

In some embodiments, the method 1100 further includes receiving user input, such as user feedback on the accuracy of the identified potential cause. For instance, a user (e.g., a clinician) can be prompted to provide input indicating whether they believe the potential cause identified by the software algorithm is accurate (e.g., after inspecting the patient's intraoral cavity to confirm the potential cause). The user can optionally provide input indicating what the user predicts is the actual cause, and the user-predicted cause can be compared to the potential cause identified by the algorithm. If there is a discrepancy between the user-predicted cause and the identified potential cause, the identified potential cause can be corrected. The user-predicted cause and/or the discrepancy can be stored in any suitable data store, such as a database, dental record, training dataset, etc. The stored user-predicted cause and/or discrepancy may subsequently be retrieved and displayed to a user when generating future identifications of potential causes of abnormal periodontal pocket depth values for the patient, e.g., to provide a reference for evaluating the accuracy of the identification and/or to allow for correction of future identifications. Optionally, the user-predicted cause and/or the discrepancy can be used to adjust (e.g., retrain) the software algorithm(s) used in the method 1100 (e.g., the factor identification algorithm 1212 of FIG. 12B and/or the cause prediction algorithm 1216 of FIG. 12C) to provide more accurate, patient-specific identifications.

### D. Multi-Modal Input Data

In some embodiments, the present technology uses data from multiple measurement modalities to evaluate a patient's intraoral health (also referred to herein as “multi-modal input data”). The multi-modal input data may include, for example, scan data obtained via intraoral scanning (e.g., surface topography data, color image data, NIR image data) as well as additional data different from the scan data, such as data obtained via other measurement techniques, data that is determined based on scan data but that is presented in a different format than the scan data (e.g., values, metrics, statistics, etc., derived from scan data), and/or data than is obtained from a different source than the scan data (e.g., data from dental records, electronic health records, user input, etc.). For example, the additional data can include x-ray data, bone height and/or bone loss data, gum recession data, inflammation data, tooth mobility data, and/or occlusion data. The use of multi-modal input data can allow for more accurate predictions of intraoral conditions and/or for predictions of many different types of intraoral conditions (e.g., not limited to conditions associated with periodontal disease). Intraoral conditions that may be predicted using multi-modal input data include, for example, periodontal disease, deep periodontal pockets, bruxism, tooth decay, and dental plaque. Moreover, multi-modal input data can be used to generate an oral health metric that summarizes the overall oral health of a particular tooth, of a particular group of teeth (e.g., sextant, quadrant), or of an entire dental arch.

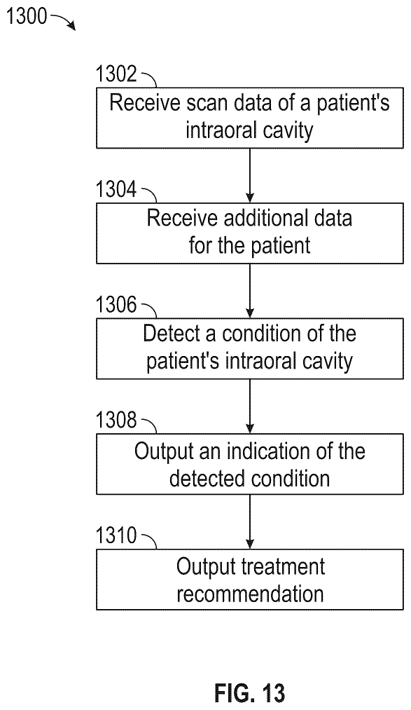

FIG. 13 is a flow diagram illustrating a method 1300 for evaluating a patient's intraoral health, in accordance with embodiments of the present technology. The method 1300 can be implemented using any of the systems and devices described herein. In some embodiments, some or all of the processes of the method 1300 are implemented as computer-readable instructions (e.g., program code) that are configured to be executed by one or more processors of a computing device (e.g., a client device, a server device, or suitable combinations thereof). The method 1300 may be combined with any of the other methods described herein, such as any of the methods described in Sections I.A-I.C above.

The method 1300 can begin at block 1302 with receiving scan data of a patient's intraoral cavity. The scan data can depict one or more intraoral structures of interest, such as the teeth, gingiva, palate, tongue, cheeks, etc. The scan data can include surface topography data, color image data, NIR image data, and/or any other data obtained via intraoral scanning, e.g., as discussed above with respect to FIG. 2. The scan data may be provided in any suitable format, such as a 3D digital representation (e.g., point cloud, height/depth map, surface model, mesh model), a 2D digital representation (e.g., 2D images, continuous texture map), or a combination thereof.

At block 1304, the method 1300 can include receiving additional data for the patient. The additional data can be data associated with a different measurement modality than the scan data (e.g., non-scan data), data that is determined based on scan data but that is presented in a different format than the scan data (e.g., values, metrics, statistics, etc., derived from scan data), and/or data than is obtained from a different source than the scan data (e.g., data from dental records, electronic health records, user input, etc.). For example, the additional data can include x-ray data, bone height and/or bone loss data, gum recession data, inflammation data, tooth mobility data, and/or occlusion data. The additional data may be received from any suitable data source, such as electronic health records, databases, imaging systems, etc.

At block 1306, the method 1300 can continue with predicting a condition of the patient's intraoral cavity, based on the scan data and the additional data. The condition can be any disease or condition of the intraoral cavity or a particular tissue thereof (e.g., teeth, gingiva, ligaments, cementum, bone, palate, cheeks, tongue), such as periodontal disease, deep periodontal pockets, bruxism, tooth decay, and/or dental plaque. Alternatively or in addition, the condition can be an oral health metric that provides a quantitative and/or qualitative summary of overall oral health (e.g., a rating from 1-10; a rating of “normal,” “moderate,” or “poor”; etc.). The condition can be predicted for a particular tooth, for a particular group of teeth (e.g., sextant, quadrant), and/or for an entire dental arch.

The condition can be predicted using one or more software algorithms, such as rule-based algorithms, machine learning models, or suitable combinations thereof. Machine learning models may be trained on input data (e.g., scan data and/or additional data) and condition data (e.g., diagnoses of intraoral conditions) from a plurality of patients in order to learn correlations between the features in the input data and conditions that are present in the patient's intraoral cavity. For example, scan data and/or other image-based data (e.g., x-ray data) may be analyzed using CNNs or other deep learning neural networks that operate on images to detect the condition. The software algorithm(s) may include any of the algorithms described elsewhere herein, e.g., in Sections I.A-I.C above.

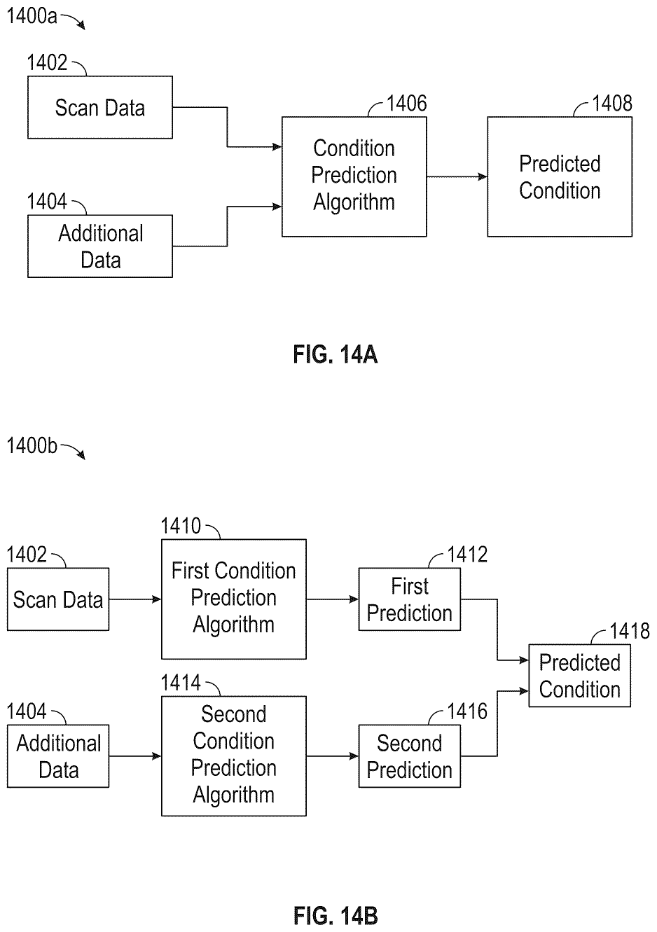

FIG. 14A is a schematic block diagram illustrating a workflow 1400a for predicting a condition of a patient's intraoral cavity, in accordance with embodiments of the present technology. As shown in FIG. 14A, multi-modal input data including scan data 1402 and additional data 1404 (e.g., non-scan data) can be provided to a condition prediction algorithm 1406 (e.g., a rule-based algorithm, a machine learning model), and the condition prediction algorithm 1406 can use the input data to determine a predicted condition 1408 of the intraoral cavity. The scan data 1402 and the additional data 1404 may be combined (e.g., concatenated) into a single input data set, e.g., according to the techniques described in connection with FIG. 6 above. This approach may be used in embodiments where a single condition prediction algorithm 1406 is used to produce predictions from multi-modal input data.

FIG. 14B is a schematic block diagram illustrating a workflow 1400b for predicting a condition of a patient's intraoral cavity, in accordance with embodiments of the present technology. In the illustrated embodiment, scan data 1402 is provided to a first condition prediction algorithm 1410 that generates a first prediction 1412 of a condition of the intraoral cavity, and additional data 1404 (e.g., non-scan data) is provided to a second condition prediction algorithm 1414 that generates a second prediction 1416 of a condition of the intraoral cavity. This approach may be used in embodiments where each algorithm is configured to generate predictions from a particular type of input data (e.g., data obtained from a single measurement modality). In such embodiments, the first condition prediction algorithm 1410 and the second condition prediction algorithm 1414 may be different types of software algorithms, e.g., the first condition prediction algorithm 1410 can be a machine learning model and the second condition prediction algorithm 1414 may be a rule-based algorithm, or vice-versa; the first condition prediction algorithm 1410 can be a first type of machine learning model and the second condition prediction algorithm 1414 can be a second type of machine learning model; the first condition prediction algorithm 1410 can be a first type of rule-based algorithm and the second condition prediction algorithm 1414 can be a second type of rule-based algorithm; etc.

The first prediction 1412 and the second prediction 1416 can be combined to generate a predicted condition 1418 for the patient's intraoral cavity. The combination can be performed in various ways, such as summing, averaging, etc. For instance, the predicted condition 1418 may be whichever one of the first prediction 1412 or the second prediction 1416 has the higher confidence score (e.g., only a single prediction is considered valid). As another example, the predicted condition 1418 can include whichever prediction has a confidence score exceeding a threshold value (e.g., both predictions may be considered valid if their confidence scores are sufficiently high). In a further example, the predicted condition 1418 is made only if the first prediction 1412 and the second prediction 1416 are consistent with each other (e.g., the predictions are considered valid only if they identify the same condition as being present and if their confidence scores both exceed a threshold value, which may be lower than the threshold value for validating a single prediction). In yet another example, the first prediction 1412 and the second prediction 1416 may be combined via averaging (e.g., with weighting based on the confidence scores) to determine the predicted condition 1418.

As a further example, the first prediction 1412 and the second prediction 1416 may be predictions of different respective diseases or conditions of the intraoral cavity, and the predicted condition 1418 can be an oral health metric that is representative of overall oral health, taking the first and second predictions 1412, 1416 into account. In some embodiments, the first prediction 1412 is a periodontal parameter (e.g., a periodontal pocket depth value), the second prediction 1416 is a different intraoral parameter than the periodontal parameter (e.g., tooth mobility, gum recession, inflammation, bone height and/or bone loss), and the predicted condition 1418 is an oral health metric that summarizes the overall health for a particular tooth, group of teeth, or the entire dental arch based on the periodontal parameter and the different intraoral parameter. For example, the oral health metric may be “poor” if both parameters indicate that a respective disease or abnormal condition is present, “good” or “normal” if both parameters indicate that a respective disease or abnormal condition is not present, and “moderate” if one parameter indicates that a first disease or abnormal condition is present and the other parameter indicates that a second disease or abnormal condition is not present. Optionally, the parameters may be weighted differently in determining the oral health metric, e.g., a periodontal parameter may be weighted more heavily than other intraoral parameters, or vice-versa. This approach may be advantageous, for example, if the individual predictions are not necessarily highly accurate on their own, but the aggregated predictions still provide a useful overall picture of whether certain areas of the intraoral cavity are or are not at risk for periodontal disease and/or other intraoral conditions.

Although FIG. 14B illustrates two condition prediction algorithms that generate two separate predictions, the workflow 1400b can be modified to incorporate any suitable number of condition prediction algorithms (e.g., three, four, five, or more), each of which operates on a respective set of input data (e.g., single modality data) to produce a respective prediction. The predictions produced by the condition prediction algorithms may be combined with each other in any suitable manner to determine the predicted condition 1418.

Referring again to FIG. 13, the process of block 1306 can include predicting a single condition or multiple conditions for the patient's intraoral cavity. Each predicted condition may optionally be associated with a confidence score, which may be a numerical value (e.g., a percentage or probability value), a quantitative assessment (e.g., “high confidence,” “low confidence”), or a combination thereof that represents the estimated degree of accuracy of the predicted condition. The confidence score may be produced by the same algorithm that determines the potential cause (e.g., the condition prediction algorithm 1406 of FIG. 14A, the first condition prediction algorithm 1410 and/or the second condition prediction algorithm 1414 of FIG. 14B). In embodiments where multiple predictions of the condition are produced, the predictions may be ranked and/or selected based on confidence score. For example, a predicted condition may be identified as such only if the associated confidence score is sufficiently high (e.g., above a threshold value). As another example, the predicted conditions produced by the algorithm(s) may be ranked based on their confidence scores, and only the condition(s) having the highest score(s) may be selected.

At block 1308, the method 1300 can include outputting an indication of the predicted condition on a display. The indication of the predicted condition can be presented in many different formats, such as numerically, textually, and/or graphically. In some embodiments, one or more indicators are overlaid onto or otherwise displayed together with a digital representation of the teeth, where the indicators show the type and/or location of the predicted condition. The predicted condition may also be displayed together with its associated confidence score, if available. In embodiments where multiple predicted conditions are available, the confidence scores may be used to select a subset of the predicted conditions for display (e.g., only the predicted condition(s) having the highest score(s) are displayed) and/or the predicted conditions may be displayed in ranked order based on confidence score.

For example, in embodiments where the predicted condition is an oral health metric, the oral health metric can be displayed to a user quantitively (e.g., as a rating from 1-10 or any other suitable scale), qualitatively (e.g., “good/normal,” “moderate,” “poor”), or suitable combinations thereof. The oral health metric can be presented as an indicator (e.g., text, graphics, coloring, shading) that is overlaid onto or otherwise displayed together with a digital representation of the teeth. Optionally, a user may click on or otherwise select the displayed indicator to view more details on the predictions that contributed to the oral health metric. For instance, in embodiments where the oral health metric provides a summary of the oral health of a group of multiple teeth, selection of the oral health metric may allow the user to view oral health metrics for each individual tooth within that group. As another example, selection of the oral health metric can allow the user to view the individual parameters that were used to determine the oral health metric, such as periodontal parameters and/or intraoral parameters such as tooth mobility, gum recession, inflammation, bone height and/or bone loss, etc. This approach may be advantageous, for example, to provide a high level overview of oral health that avoids overwhelming the user with information, while also allowing the user to view more details on specific regions and/or parameters of interest if desired.

At block 1310, the method 1300 can optionally include outputting a treatment recommendation, based on the predicted condition. The treatment recommendation can include one or more actions to be performed by the clinician and/or patient to address the predicted condition. Treatment recommendations that may be provided include, for example, performing additional diagnostic procedures to confirm the predicted condition (e.g., visual inspection, physical probing, x-rays) and/or performing appropriate therapeutic interventions. For example, an oral health metric may be used as a screening tool to flag high risk areas where additional diagnostic procedures are recommended, e.g., a poor oral health metric for a particular tooth may indicate that additional diagnostics should be performed for that tooth and/or nearby teeth (e.g., teeth within the same sextant or quadrant).

In some embodiments, the method 1300 further includes receiving user input, such as user feedback on the accuracy of the predicted condition. For instance, a user (e.g., a clinician) can be prompted to provide input indicating whether they believe the predicted condition is accurate (e.g., after inspecting the patient's intraoral cavity to confirm the condition). The user can optionally provide input indicating what the user identifies as the actual condition of the intraoral cavity, and the user-identified condition can be compared to the predicted condition. If there is a discrepancy between the user-identified condition and the predicted condition, the predicted condition can be corrected. The user-identified condition and/or the discrepancy can be stored in any suitable data store, such as a database, dental record, training dataset, etc. The stored user-identified prediction and/or discrepancy may subsequently be retrieved and displayed to a user when generating future predictions of the condition of the intraoral cavity, e.g., to provide a reference for evaluating the accuracy of the future predictions and/or to allow for correction of future predictions. Optionally, the user-identified condition and/or the discrepancy can be used to adjust (e.g., retrain) the software algorithm(s) used in the method 1300 (e.g., the condition prediction algorithm 1406 of FIG. 14A, the first condition prediction algorithm 1410 and/or the second condition prediction algorithm 1414 of FIG. 14B) to provide more accurate, patient-specific predictions.

### II. Overview of Intraoral Scanning Technology

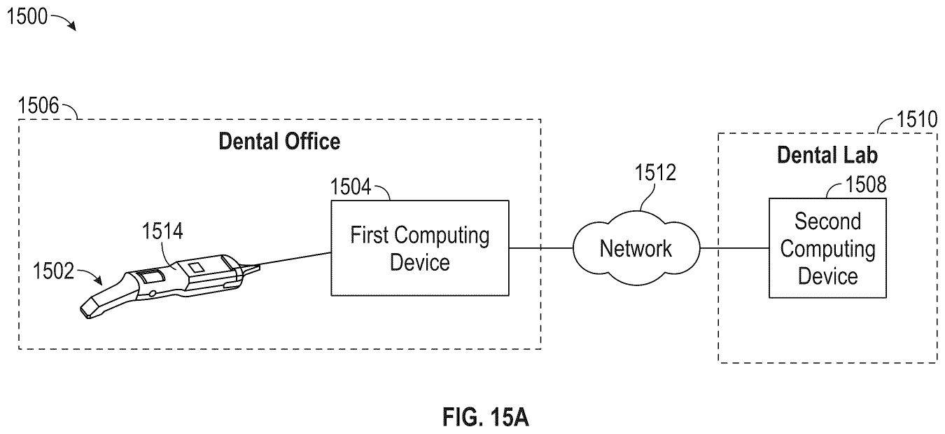

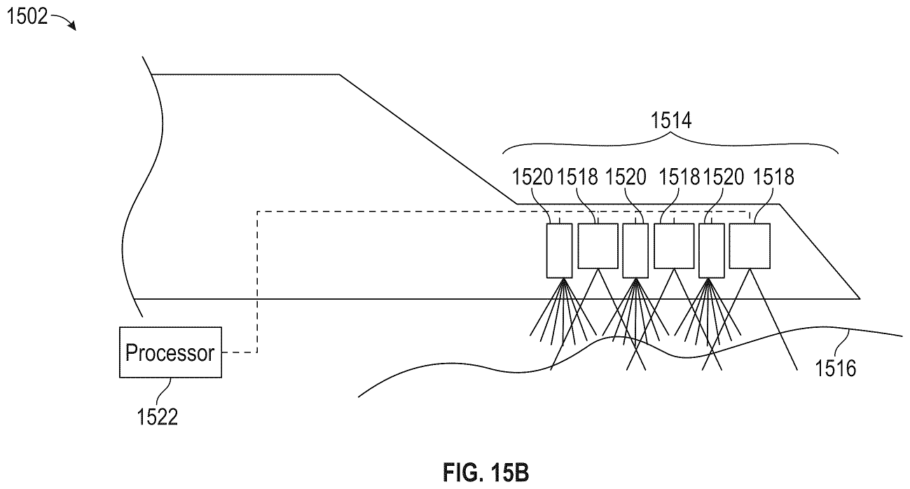

FIG. 15A schematically illustrates a system 1500 for performing intraoral scanning and/or generating 3D digital representations of a patient's intraoral cavity, in accordance with embodiments of the present technology. The system 1500 may be used to generate scan data for use in any of the methods described herein (e.g., in Section I above). The system 1500 includes an intraoral scanner 1502 (also referred to as a “scanner”) operably coupled to a first computing device 1504. The scanner 1502 and first computing device 1504 can be at a first location, such as a dental office 1506. Optionally, the first computing device 1504 may be operably coupled to another second computing device 1508 at a second location, such as a dental lab 1510. The first computing device 1504 and the second computing device 1508 can be connected to one another via a network 1512, such as a local area network (LAN), a public wide area network (WAN) (e.g., the Internet), a private WAN (e.g., an intranet), or a combination thereof.

The scanner 1502 may be used to generate scan data of one or more intraoral structures of a patient, such as the teeth, gingiva, palate, tongue, cheeks, etc. The scan data can include 3D intraoral scans that provide a digital representation of the surface topography of the intraoral structures. For instance, the 3D intraoral scans can include one or more point clouds, height/depth maps, or any other suitable digital data format depicting the 3D geometry of the intraoral structures. Optionally, the scan data can include other types of digital data, such as color images and/or images obtained at various wavelengths (e.g., near-infrared (NIR) images, infrared images, ultraviolet images, etc.). In some embodiments, the scanner 1502 alternates between generation of 3D intraoral scans and one or more types of 2D intraoral images (e.g., color images, NIR images, infrared images, ultraviolet images) during scanning.

In some embodiments, the scanner 1502 includes a probe 1514 (e.g., a handheld wand) that may be inserted at least partially into the intraoral cavity. The probe 1514 can include or be coupled to one or more optical elements for outputting light toward intraoral structures and optically capturing features (e.g., surface topography, color) of the intraoral structures, such as one or more imaging devices (e.g., cameras), light sources (e.g., projectors, lasers), image sensors (e.g., CCD sensors, CMOS sensors), focusing optics (e.g., confocal optics), mirrors, prisms, lenses, beam splitters, polarizers, etc. The probe 1514 can include a transparent or translucent window to allow light to pass out of the probe 1514 toward the intraoral structures, and to allow light from the intraoral structures to be received by the probe 1514.

FIG. 15B is a partially schematic illustration of an example scanner 1502 that may be used in the system 1500 of FIG. 15A, in accordance with embodiments of the present technology. The scanner 1502 can be used to obtain scan data of an intraoral surface 1516. In some embodiments, the scanner 1502 includes a probe 1514 at a distal end of the scanner 1502. One or more cameras 1518 are disposed within the probe 1514 (e.g., rigidly fixed within the probe 1514) and arranged within the probe 1514 such that the cameras 1518 receive rays of light from an intraoral cavity in a non-central manner (e.g., the relationship between points in 3D world-coordinate space and corresponding points on the camera sensors of the one or more cameras 1518 is described by a set of camera rays for which there is no single point in space through which all of the camera rays pass).

In some embodiments, the scanner 1502 is configured to perform intraoral scanning using structured light illumination. In such embodiments, one or more structured light projectors 1520 can be disposed within the probe 1514 and can project a pattern of structured light (e.g., a pattern of spots) onto the intraoral surface 1516. Each camera 1518 can be configured to capture a plurality of images that depict at least a portion of the projected pattern of structured light on the intraoral surface 1516. In some embodiments, the structured light projectors 1520 and cameras 1518 are arranged in a closely packed and/or alternating fashion, such that a substantial part of each camera's field of view overlaps the field of view of neighboring cameras 1518, and a substantial part of each projector's field of illumination overlaps the field of illumination of neighboring projectors 1520. The positioning of the projectors 1520 and the cameras 1518 within the probe 1514 can allow the scanner 1502 to have an overall large field of view while maintaining a low profile probe geometry.

The scanner 1502 can further include a processor 1522 configured to generate a 3D model of the intraoral surface 1516 based on images from one or more cameras 1518. In some embodiments, the processor 1522 solves a “correspondence problem,” where a correspondence between pattern elements in the structured light pattern and pattern elements seen by a camera 1518 viewing the pattern is determined. The processor 1522 may compensate for the image distortion specifically introduced by the non-central manner in which one or more cameras 1518 receive rays of light from the intraoral surface 1516 by altering the coordinates of one or more of the structured light pattern elements as seen by one or more cameras 1518 in order to account for the non-central manner in which the one or more cameras 1518 receive rays of light from the intraoral surface 1516.

Referring again to FIG. 15A, other types of scanners 1502 can be used in the system 1500, alternatively or in addition to the embodiment of FIG. 15B. For instance, in some embodiments, the scanner 1502 can be a confocal imaging apparatus including a light source that emits an array of light beams. The light source can be located at a proximal end of the probe 1514, and the probe 1514 can define a light transmission path from the proximal end of the probe 1514 to the distal end of the probe. A set of confocal focusing optics may be positioned along the light transmission path between the proximal end and distal end of the probe 1514. At the distal end, the probe 1514 can include a mirror that directs the array of light beams towards an object outside of the scanner 1502. The light beams reflected off the object can pass back into the probe 1514 and be directed onto an image sensor. In some embodiments, the image sensor detects light intensity at each pixel, which may be used to compute height or depth.

Optionally, the scanner 1502 may include other components, such as a movement sensor for measuring movement and/or pose of the scanner 1502. For example, the movement sensor can be an internal measurement unit (IMU) (e.g., a micro-electromechanical system (MEMS) IMU), which may include one or more accelerometers, gyroscopes, magnetometers, pressure sensors, etc. As another example, the scanner 1502 can include a temperature sensor and temperature control circuitry for measuring and controlling the temperature within the probe 1514, e.g., to reducing fogging of optical elements and/or avoid patient discomfort. In a further example, the scanner 1502 may be used in conjunction with a removable sleeve or other protective device that fits over the probe 1514 to avoid contamination and/or for patient protection. The sleeve may be a single-use component or may be reusable.

Representative examples of intraoral scanners that may be used as the scanner 1502 are described in U.S. Pat. Nos. 11,563,929 and 11,896,461, the disclosures of each of which are incorporated by reference herein in their entirety.

The scanner 1502 can be coupled to the first computing device 1504 via a wired or wireless connection. In some embodiments, the scanner 1502 is wirelessly connected to the first computing device 1504 via a direct wireless connection. In some embodiments, the scanner 1502 is wirelessly connected to the first computing device 1504 via a wireless network, such as a Wi-Fi network, Bluetooth network, a Zigbee network, or other wireless network. For example, the first computing device 1504 may be physically connected to one or more wireless access points and/or wireless routers (e.g., Wi-Fi access points/routers), and the scanner 1502 may include a wireless module (e.g., a Wi-Fi module) for joining the wireless network via the wireless access point and/or router.

The scan data obtained by the scanner 1502 may be transmitted to the first computing device 1504, and the first computing device 1504 may store the scan data in a data store. The data store may include local data stores and/or remote data stores. The first computing device 1504 can be a personal computer, workstation, laptop, tablet, smartphone, etc., that includes one or more processors, memory, secondary storage devices, input devices (e.g., a keyboard, mouse, tablet, touchscreen, microphone, camera), output devices (e.g., display, printer, touchscreen, speakers), and/or other suitable hardware components. The first computing device 1504 can also include software components for monitoring and controlling the scanner 1502, receiving and processing the scan data, and/or other functionality relevant to an intraoral scanning procedure.

In some embodiments, a user (e.g., a patient, a clinician, technician, or other practitioner) performs intraoral scanning of a patient in connection with a dental procedure. By way of non-limiting example, dental procedures may be broadly divided into prosthodontic (restorative) and orthodontic procedures, and then further subdivided into specific forms of these procedures. Additionally, dental procedures may include identification and treatment of periodontal disease, sleep apnea, and intraoral conditions. The term prosthodontic procedure may refer to any procedure involving the oral cavity and directed to the design, manufacture, or installation of a dental prosthesis at a dental site within the oral cavity, or a real or virtual model thereof, or directed to the design and preparation of the dental site to receive such a prosthesis. A prosthesis may include any restoration such as crowns, veneers, inlays, onlays, implants and bridges, for example, and any other artificial partial or complete denture. The term orthodontic procedure may refer to any procedure involving the intraoral cavity and directed to the design, manufacture, or installation of orthodontic elements at a dental site within the intraoral cavity, or a real or virtual model thereof, or directed to the design and preparation of the dental site to receive such orthodontic elements. These elements may be appliances including but not limited to brackets and wires, retainers, aligners, palatal expanders, attachment placement templates, mouth guards, oral sleep apnea devices, or other dental appliances.

In some embodiments, intraoral scanning is performed on a patient's intraoral cavity during a visitation of the dental office 1506. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches, and may be performed in order to gather information for performing dental and/or periodontal diagnostics, to generate a treatment plan, to determine progress of a treatment plan, and/or for other purposes.

During an intraoral scanning procedure, the user may apply the scanner 1502 to one or more locations within the intraoral cavity of the patient. The scanning may be divided into one or more segments. As an example, the segments may include a lower dental arch of the patient (e.g., the complete lower dental arch or a portion thereof), an upper dental arch of the patient (e.g., the complete upper dental arch or a portion thereof), and/or patient bite (e.g., scanning performed with closure of the patient's mouth with the scan being directed towards an interface area of the patient's upper and lower teeth). Via such scanner application, the scanner 1502 may provide scan data to the first computing device 1504. The scan data may be provided in the form of intraoral scan data sets, each of which may include 3D intraoral scans (e.g., point clouds, height/depth maps) and/or 2D intraoral images (e.g., color images, NIR images, infrared images, ultraviolet images).

The first computing device 1504 can include one or more software components configured to process the scan data into a 3D digital representation of the patient's intraoral structures. For example, the first computing device 1504 can implement an intraoral scan application that registers and stitches together two or more intraoral scans from the scan data to generate a growing 3D surface. In some embodiments, performing registration includes capturing 3D data of various points of a surface in multiple scans, and registering the scans by computing transformations between the scans (e.g., based on overlapping points depicted in the scans). One or more 3D surfaces may be generated based on the registered and stitched together intraoral scans during the intraoral scanning. The one or more 3D surfaces may be output to a graphical user interface (GUI) on a display of the first computing device 1504 so that the user can view the scan progress thus far. As each new intraoral scan is captured and registered to previous intraoral scans and/or to the generated 3D surface(s), the 3D surface(s) may be updated, and the updated 3D surface(s) may be output to the display. The user interface showing the 3D surface(s) may be periodically or continuously updated to show scanning progress in real time or near-real time.

When a scan session or a portion of a scan session associated with a particular scanning segment (e.g., upper dental arch, lower dental arch, bite) is complete (e.g., all scans for the site of interest have been captured), the intraoral scan application may generate a 3D digital representation of the scanned segment (e.g., a virtual 3D model). The 3D digital representation may be a set of 3D points and their connections with each other (e.g., a mesh). To generate the 3D digital representation, the intraoral scan application may register and stitch together the intraoral scans generated from the intraoral scan session that are associated with a particular scanning segment. The registration performed at this stage may be more accurate than the registration performed during the capturing of the intraoral scans, and may take more time to complete than the registration performed during the capturing of the intraoral scans. In some embodiments, performing scan registration includes capturing 3D data of various points of a surface in multiple scans, and registering the scans by computing transformations between the scans. The 3D data may be projected into a 3D space of the 3D digital representation to form a portion of the 3D digital representation. The intraoral scans may be integrated into a common reference frame by applying appropriate transformations to points of each registered scan and projecting each scan into the 3D space.

In some embodiments, registration is performed for adjacent or overlapping intraoral scans (e.g., each successive frame of an intraoral video). Registration algorithms may be carried out to register two adjacent or overlapping intraoral scans and/or to register an intraoral scan with a 3D digital representation, which can involve determination of the transformations which align one scan with the other scan and/or with the 3D digital representation. Registration may involve identifying multiple points in each scan (e.g., point clouds) of a scan pair (or of a scan and the 3D digital representation), surface fitting to the points, and using local searches around points to match points of the two scans (or of the scan and the 3D digital representation). For example, the intraoral scan application may match points of one scan with the closest points interpolated on the surface of another scan, and iteratively minimize the distance between matched points. Other registration techniques known to those of skill in the art may also be used. Examples of registration techniques include, for example, iterative closest point (ICP) algorithms.

The intraoral scan application may repeat registration for all intraoral scans of a sequence of intraoral scans to obtain transformations for each intraoral scan, to register each intraoral scan with previous intraoral scan(s) and/or with a common reference frame (e.g., with the 3D digital representation). Intraoral scan application may integrate intraoral scans into a single 3D digital representation by applying the appropriate determined transformations to each of the intraoral scans. Each transformation may include rotations about one to three axes and/or translations along one to three axes.

The intraoral scan application may generate one or more 3D digital representations from intraoral scans, and may display the 3D digital representation(s) to the user via a GUI on the display. The 3D digital representation(s) can then be checked visually by the user. The user can virtually manipulate the 3D digital representation(s) via the user interface with respect to up to six degrees of freedom (e.g., translated and/or rotated with respect to one or more of three mutually orthogonal axes) using suitable user controls (e.g., hardware and/or software controls) to enable viewing of the 3D digital representation(s) from any desired direction.

Optionally, the scan data generated by the scanner 1502 and/or the 3D digital representation(s) generated by the intraoral scan application may be transmitted from the first computing device 1504 to the second computing device 1508 via the network 1512. The second computing device 1508 can be coupled to a data store for storing the scan data and/or the 3D digital representations, which may include local data stores and/or remote data stores. The second computing device 1508 can be a personal computer, workstation, laptop, tablet, smartphone, etc., that includes one or more processors, memory, secondary storage devices, input devices (e.g., a keyboard, mouse, tablet, touchscreen, microphone, camera), output devices (e.g., display, printer, touchscreen, speakers), and/or other suitable hardware components.

In some embodiments, the second computing device 1508 includes one or more software components configured to perform dental and/or periodontal diagnostics, generate a treatment plan, to determine progress of a treatment plan, and/or for other purposes relevant to a dental procedure, based on the scan data and/or the 3D digital representation(s). For example, a 3D digital representation of a patient's intraoral cavity may be used to design a dental prosthesis for a prosthodontic procedure, such as one or more crowns, veneers, inlays, onlays, implants, bridges, etc. As another example, a 3D digital representation of a patient's intraoral cavity may be used to design a dental appliance for an orthodontic procedure, such as one more aligners, retainers, palatal expanders, etc. In a further example, a 3D digital representation of a patient's intraoral cavity may be used to diagnose a patient with periodontal disease, sleep apnea, and/or other intraoral conditions.

In some embodiments, one or more 3D digital representations can be used for designing dental appliances, such as aligners and/or a series of aligners with tooth-receiving cavities configured to move a person's teeth from an initial arrangement toward a target arrangement in accordance with a treatment plan. Aligners can include mandibular repositioning elements, such as those described in U.S. Pat. No. 10,912,629, entitled “Dental Appliances with Repositioning Jaw Elements,” filed Nov. 30, 2015; U.S. Pat. No. 10,537,406, entitled “Dental Appliances with Repositioning Jaw Elements,” filed Sep. 19, 2014; and U.S. Pat. No. 9,844,424, entitled “Dental Appliances with Repositioning Jaw Elements,” filed Feb. 21, 2014; all of which are incorporated by reference herein in their entirety.

One or more 3D digital representations can also be used to design attachment placement devices, e.g., appliances used to position prefabricated attachments on a person's teeth in accordance with one or more aspects of a treatment plan. Examples of attachment placement devices (also known as “attachment placement templates” or “attachment fabrication templates”) can be found at least in: U.S. application Ser. No. 17/249,218, entitled “Flexible 3D Printed Orthodontic Device,” filed Feb. 24, 2021; U.S. application Ser. No. 16/366,686, entitled “Dental Attachment Placement Structure,” filed Mar. 27, 2019; U.S. application Ser. No. 15/674,662, entitled “Devices and Systems for Creation of Attachments,” filed Aug. 11, 2017; U.S. Pat. No. 11,103,330, entitled “Dental Attachment Placement Structure,” filed Jun. 14, 2017; U.S. application Ser. No. 14/963,527, entitled “Dental Attachment Placement Structure,” filed Dec. 9, 2015; U.S. application Ser. No. 14/939,246, entitled “Dental Attachment Placement Structure,” filed Nov. 12, 2015; U.S. application Ser. No. 14/939,252, entitled “Dental Attachment Formation Structures,” filed Nov. 12, 2015; and U.S. Pat. No. 9,700,385, entitled “Attachment Structure,” filed Aug. 22, 2014; all of which are incorporated by reference herein in their entirety.

One or more 3D digital representations can be used to design incremental palatal expanders and/or a series of incremental palatal expanders used to expand a person's palate from an initial position toward a target position in accordance with one or more aspects of a treatment plan. Examples of incremental palatal expanders can be found at least in: U.S. application Ser. No. 16/380,801, entitled “Releasable Palatal Expanders,” filed Apr. 10, 2019; U.S. application Ser. No. 16/022,552, entitled “Devices, Systems, and Methods for Dental Arch Expansion,” filed Jun. 28, 2018; U.S. Pat. No. 11,045,283, entitled “Palatal Expander with Skeletal Anchorage Devices,” filed Jun. 8, 2018; U.S. application Ser. No. 15/831,159, entitled “Palatal Expanders and Methods of Expanding a Palate,” filed Dec. 4, 2017; U.S. Pat. No. 10,993,783, entitled “Methods and Apparatuses for Customizing a Rapid Palatal Expander,” filed Dec. 4, 2017; and U.S. Pat. No. 7,192,273, entitled “System and Method for Palatal Expansion,” filed Aug. 7, 2003; all of which are incorporated by reference herein in their entirety.

The system 1500 can be configured in many different ways. For example, any of the components of the system 1500 shown as distinct elements in FIG. 15A can be combined into a single device, and/or any of the components of the system 1500 shown as a single element in FIG. 15A can be divided into a plurality of discrete devices. Moreover, the locations of the components of the system 1500 can be varied as desired, e.g., any of the components shown in FIG. 15A can be located at the dental office 1506, the dental lab 1510, or at one or more other locations, such as a server farm that provides a cloud computing service, a facility of a manufacturer of the scanner 1502, a facility of a manufacturer of dental appliances and/or dental prosthetics, etc. Additionally, any of the operations that are described as being performed by a particular component of the system 1500 can alternatively or additionally be performed by any other component of the system 1500, e.g., the operations of the first computing device 1504 may alternatively or additionally be performed by the second computing device 1508 and/or by another computing device (e.g., a remote server), and vice-versa.

The system 1500 may include additional components not illustrated in FIG. 15A. For instance, although FIG. 15A depicts a single scanner 1502, the system 1500 can optionally include multiple scanners 1502, which may be at the same location (e.g., the same dental office 1506) or at different locations (e.g., different dental offices 1506). Similarly, although FIG. 15A depicts a single dental office 1506 and a single dental lab 1510, the system 1500 may include multiple dental offices 1506, multiple dental labs 1510, and/or other facilities including respective computing devices that are communicably coupled to each other via one or more networks 1512 in any suitable arrangement.

### EXAMPLES

The following examples are included to further describe some aspects of the present technology, and should not be used to limit the scope of the technology.

Example 1. A system for evaluating a patient's intraoral health, the system comprising:
one or more processors; and
a memory operably coupled to the one or more processors and storing instructions that, when executed by the one or more processors, cause the system to perform operations comprising:
   receiving scan data of a patient's intraoral cavity;
   receiving additional data for the patient, the additional data being different from the scan data;
   determining a first prediction of a condition of the intraoral cavity based on the scan data;
   determining a second prediction of a condition of the intraoral cavity based on the additional data;
   generating a predicted condition for the intraoral cavity based on a combination of the first and second predictions; and
   outputting an indication of the predicted condition on a display.

Example 2. The system of Example 1, wherein the additional data comprises one or more of demographic information, health condition data, previous periodontal parameter data, palate color data, other imaging modality data, bone height data, bone loss data, gum recession data, inflammation data, tooth mobility data, or occlusion data.

Example 3. The system of Example 1 or 2, wherein the first prediction comprises a periodontal parameter, and the second prediction comprises an intraoral parameter different from the periodontal parameter.

Example 4. The system of Example 3, wherein the periodontal parameter comprises a periodontal pocket depth value.

Example 5. The system of Example 3 or 4, wherein the intraoral parameter is indicative of one or more of tooth mobility, gum recession, inflammation, bone height, or bone loss.

Example 6. The system of any one of Examples 1 to 5, wherein the predicted condition comprises an oral health metric.

Example 7. The system of Example 6, wherein the oral health metric is representative of overall oral health for a plurality of teeth.

Example 8. The system of any one of Examples 1 to 7, wherein the first prediction is determined using a first condition prediction algorithm, and wherein the second prediction is determined using a second condition prediction algorithm different from the first condition prediction algorithm.

Example 9. The system of any one of Examples 1 to 8, wherein the indication is displayed together with a digital representation of the patient's teeth.

Example 10. The system of any one of Examples 1 to 9, wherein the operations further comprise outputting a treatment recommendation on the display, and wherein the treatment recommendation comprises performing an additional diagnostic procedure at a location associated with the predicted condition.

Example 11. A computer-implemented method for evaluating a patient's intraoral health, the computer-implemented method comprising:
receiving scan data of a patient's intraoral cavity;
receiving additional data for the patient, the additional data being different from the scan data;
determining a first prediction of a condition of the intraoral cavity based on the scan data;
determining a second prediction of a condition of the intraoral cavity based on the additional data;
generating a predicted condition for the intraoral cavity based on a combination of the first and second predictions; and
outputting an indication of the predicted condition on a display.

Example 12. The computer-implemented method of Example 11, wherein the additional data comprises one or more of demographic information, health condition data, previous periodontal parameter data, palate color data, other imaging modality data, bone height data, bone loss data, gum recession data, inflammation data, tooth mobility data, or occlusion data.

Example 13. The computer-implemented method of Example 11 or 12, wherein the first prediction comprises a periodontal parameter, and the second prediction comprises an intraoral parameter different from the periodontal parameter.

Example 14. The computer-implemented method of Example 13, wherein the periodontal parameter comprises a periodontal pocket depth value.

Example 15. The computer-implemented method of Example 13 or 14, wherein the intraoral parameter is indicative of one or more of tooth mobility, gum recession, inflammation, bone height, or bone loss.

Example 16. The computer-implemented method of any one of Examples 11 to 15, wherein the predicted condition comprises an oral health metric.

Example 17. The computer-implemented method of Example 16, wherein the oral health metric is representative of overall oral health for a plurality of teeth.

Example 18. The computer-implemented method of any one of Examples 11 to 17, wherein the first prediction is determined using a first condition prediction algorithm, and wherein the second prediction is determined using a second condition prediction algorithm different from the first condition prediction algorithm.

Example 19. The computer-implemented method of any one of Examples 11 to 18, wherein the indication is displayed together with a digital representation of the patient's teeth.

Example 20. The computer-implemented method of any one of Examples 11 to 19, further comprising outputting a treatment recommendation on the display, wherein the treatment recommendation comprises performing an additional diagnostic procedure at a location associated with the predicted condition.

Example 21. A system for evaluating a patient's intraoral health, the system comprising:
one or more processors; and
a memory operably coupled to the one or more processors and storing instructions that, when executed by the one or more processors, cause the system to perform operations comprising:
   determining, based on scan data of a patient's intraoral cavity, a periodontal parameter for one or more teeth of the intraoral cavity;
   determining, based on additional data for the patient, an intraoral parameter for the one or more teeth, the intraoral parameter being different from the periodontal parameter;
   generating based on the periodontal parameter and the intraoral parameter, an oral health metric for the one or more teeth;
   outputting, on a display device, the oral health metric together with a digital representation of the one or more teeth; and
   outputting, on the display device, in response to user input selecting the oral health metric, one or more of the periodontal parameter or the intraoral parameter.

Example 22. The system of Example 21, wherein the additional data comprises one or more of demographic information, health condition data, previous periodontal parameter data, palate color data, other imaging modality data, bone height data, bone loss data, gum recession data, inflammation data, tooth mobility data, or occlusion data.

Example 23. The system of Example 21 or 22, wherein the periodontal parameter comprises a periodontal pocket depth value.

Example 24. The system of any one of Examples 21 to 23, wherein the intraoral parameter is indicative of one or more of tooth mobility, gum recession, inflammation, bone height, or bone loss.

Example 25. The system of any one of Examples 21 to 24, wherein the oral health metric is representative of overall oral health for a plurality of teeth.

Example 26. A computer-implemented method for evaluating a patient's intraoral health, the computer-implemented method comprising:
determining, based on scan data of a patient's intraoral cavity, a periodontal parameter for one or more teeth of the intraoral cavity;
determining, based on additional data for the patient, an intraoral parameter for the one or more teeth, the intraoral parameter being different from the periodontal parameter;
generating based on the periodontal parameter and the intraoral parameter, an oral health metric for the one or more teeth;
outputting, on a display device, the oral health metric together with a digital representation of the one or more teeth; and
outputting, on the display device, in response to user input selecting the oral health metric, one or more of the periodontal parameter or the intraoral parameter.

Example 27. The computer-implemented method of Example 26, wherein the additional data comprises one or more of demographic information, health condition data, previous periodontal parameter data, palate color data, other imaging modality data, bone height data, bone loss data, gum recession data, inflammation data, tooth mobility data, or occlusion data.

Example 28. The computer-implemented method of Example 26 or 27, wherein the periodontal parameter comprises a periodontal pocket depth value.

Example 29. The computer-implemented method of any one of Examples 26 to 28, wherein the intraoral parameter is indicative of one or more of tooth mobility, gum recession, inflammation, bone height, or bone loss.

Example 30. The computer-implemented method of any one of Examples 26 to 29, wherein the oral health metric is representative of overall oral health for a plurality of teeth.

Example 31. A system for evaluating a patient's intraoral health, the system comprising:
one or more processors; and
a memory operably coupled to the one or more processors and storing instructions that, when executed by the one or more processors, cause the system to perform operations comprising:
   receiving scan data of a patient's intraoral cavity;
   receiving additional data representing one or more patient-specific characteristics of the patient;
   determining, based on the scan data and additional data, a periodontal parameter indicative of a periodontal condition of the patient, wherein the periodontal parameter is determined using a machine learning model that is trained on scan data and periodontal parameter measurements from a plurality of patients, and wherein the machine learning model is configured to use the additional data to account for the one or more patient-specific characteristics when determining the periodontal parameter; and
   outputting an indication of the periodontal parameter on a display.

Example 32. The system of Example 31, wherein the additional data comprises one or more of demographic information, health condition data, previous periodontal parameter data, palate color data, other imaging modality data, bone height data, bone loss data, gum recession data, inflammation data, tooth mobility data, or occlusion data.

Example 33. The system of Example 32, wherein the additional data comprises the previous periodontal parameter data, and the previous periodontal parameter data comprises one or more previous periodontal pocket depth values.

Example 34. The system of Example 33, wherein the one or more previous periodontal pocket depth values are for only a subset of the patient's teeth.

Example 35. The system of Example 33, wherein the one or more previous periodontal pocket depth values are for all of the patient's teeth.

Example 36. The system of any one of Examples 32 to 35, wherein the additional data comprises the demographic information, and the demographic information comprises one or more of age, ethnicity, or gender.

Example 37. The system of any one of Examples 32 to 36, wherein the additional data comprises the other imaging modality data, and the other imaging modality data comprises x-ray data.

Example 38. The system of any one of Examples 31 to 37, wherein the scan data comprises one or more of surface topography data, color data, or near infrared image data.

Example 39. The system of any one of Examples 31 to 38, wherein the operations further comprise processing the scan data to generate a continuous texture map, prior to determining the periodontal parameter.

Example 40. The system of any one of Examples 31 to 39, further comprising:
generating an input data set by combining the scan data and the additional data, and
inputting the input data set into the machine learning model.

Example 41. The system of any one of Examples 31 to 40, wherein the indication comprises a predicted value for the periodontal parameter, and wherein the operations further comprise:
prompting a user for an actual value of the periodontal parameter, and
receiving user input indicative of the actual value of the periodontal parameter.

Example 42. The system of Example 41, further comprising:
comparing the predicted value of the periodontal parameter to the actual value of the periodontal parameter, and
adjusting the machine learning model based on the comparison.

Example 43. The system of Example 42, further comprising using the adjusted machine learning model to determine an additional periodontal parameter for the patient.

Example 44. The system of any one of Examples 31 to 43, further comprising:
determining whether an adjusted machine learning model that has been trained on patient-specific data for the patient is available,
if the adjusted machine learning model is available, using the adjusted machine learning model to determine the periodontal parameter, and
if the adjusted machine learning model is not available, using a generic machine learning model that has not been trained on patient-specific data for the patient to determine the periodontal parameter.

Example 45. The system of any one of Examples 31 to 44, wherein the indication comprises a periodontal pocket depth value.

Example 46. The system of Example 45, wherein the indication comprises a location associated with the periodontal pocket depth value.

Example 47. The system of any one of Examples 31 to 46, further comprising:
determining whether the periodontal parameter is indicative of an abnormal periodontal pocket depth value, and
in response to a determination that the periodontal parameter is indicative of the abnormal periodontal pocket depth value, outputting a notification to a user.

Example 48. The system of Example 47, wherein determining whether the periodontal parameter is indicative of the abnormal periodontal pocket depth value comprises comparing the periodontal parameter to a patient-specific threshold value.

Example 49. A computer-implemented method for evaluating a patient's intraoral health, the computer-implemented method comprising:
receiving scan data of a patient's intraoral cavity;
receiving additional data representing one or more patient-specific characteristics of the patient;
determining, based on the scan data and additional data, a periodontal parameter indicative of a periodontal condition of the patient, wherein the periodontal parameter is determined using a machine learning model that is trained on scan data and periodontal parameter measurements from a plurality of patients, and wherein the machine learning model is configured to use the additional data to account for the one or more patient-specific characteristics when determining the periodontal parameter; and
outputting an indication of the periodontal parameter on a display.

Example 50. The computer-implemented method of Example 49, wherein the additional data comprises one or more of demographic information, health condition data, previous periodontal parameter data, palate color data, other imaging modality data, bone height data, bone loss data, gum recession data, inflammation data, tooth mobility data, or occlusion data.

Example 51. The computer-implemented method of Example 50, wherein the additional data comprises the previous periodontal parameter data, and the previous periodontal parameter data comprises one or more previous periodontal pocket depth values.

Example 52. The computer-implemented method of Example 51, wherein the one or more previous periodontal pocket depth values are for only a subset of the patient's teeth.

Example 53. The computer-implemented method of Example 51, wherein the one or more previous periodontal pocket depth values are for all of the patient's teeth.

Example 54. The computer-implemented method of any one of Examples 50 to 53, wherein the additional data comprises the demographic information, and the demographic information comprises one or more of age, ethnicity, or gender.

Example 55. The computer-implemented method of any one of Examples 50 to 54, wherein the additional data comprises the other imaging modality data, and the other imaging modality data comprises x-ray data.

Example 56. The computer-implemented method of any one of Examples 49 to 55, wherein the scan data comprises one or more of surface topography data, color data, or near infrared image data.

Example 57. The computer-implemented method of any one of Examples 49 to 56, further comprising processing the scan data to generate a continuous texture map, prior to determining the periodontal parameter.

Example 58. The computer-implemented method of any one of Examples 49 to 57, further comprising:
generating an input data set by combining the scan data and the additional data, and
inputting the input data set into the machine learning model.

Example 59. The computer-implemented method of any one of Examples 49 to 58, wherein the indication comprises a predicted value for the periodontal parameter, and wherein the computer-implemented method further comprises:
prompting a user for an actual value of the periodontal parameter, and
receiving user input indicative of the actual value of the periodontal parameter.

Example 60. The computer-implemented method of Example 59, further comprising:
comparing the predicted value of the periodontal parameter to the actual value of the periodontal parameter, and
adjusting the machine learning model based on the comparison.

Example 61. The computer-implemented method of Example 60, further comprising using the adjusted machine learning model to determine an additional periodontal parameter for the patient.

Example 62. The computer-implemented method of any one of Examples 49 to 61, further comprising:
determining whether an adjusted machine learning model that has been trained on patient-specific data for the patient is available,
if the adjusted machine learning model is available, using the adjusted machine learning model to determine the periodontal parameter, and
if the adjusted machine learning model is not available, using a generic machine learning model that has not been trained on patient-specific data for the patient to determine the periodontal parameter.

Example 63. The computer-implemented method of any one of Examples 49 to 62, wherein the indication comprises a periodontal pocket depth value.

Example 64. The computer-implemented method of Example 63, wherein the indication comprises a location associated with the periodontal pocket depth value.

Example 65. The computer-implemented method of any one of Examples 49 to 64, further comprising:
determining whether the periodontal parameter is indicative of an abnormal periodontal pocket depth value, and
in response to a determination that the periodontal parameter is indicative of the abnormal periodontal pocket depth value, outputting a notification to a user.

Example 66. The computer-implemented method of Example 65, wherein determining whether the periodontal parameter is indicative of the abnormal periodontal pocket depth value comprises comparing the periodontal parameter to a patient-specific threshold value.

Example 67. A system for evaluating a patient's intraoral health, the system comprising:
one or more processors; and
a memory operably coupled to the one or more processors and storing instructions that, when executed by the one or more processors, cause the system to perform operations comprising:
   receiving scan data of a patient's intraoral cavity;
   determining, based on the scan data, an initial periodontal parameter indicative of a periodontal condition of the patient, wherein the initial periodontal parameter is determined using a machine learning model that is trained on scan data and periodontal parameter measurements from a plurality of patients;
   determining whether additional data representing one or more patient-specific characteristics of the patient is available;
   if the additional data is available:
      adjusting the initial periodontal parameter based on the additional data, and
      outputting an indication of the adjusted periodontal parameter on a display; and
   if the additional data is not available, outputting an indication of the initial periodontal parameter on the display.

Example 68. The system of Example 67, wherein the additional data comprises previous scan data and previous periodontal parameter data for the patient.

Example 69. The system of Example 68, wherein adjusting the initial periodontal parameter comprises:
determining a predicted periodontal parameter for the patient using the machine learning model and the previous scan data,
comparing the predicted periodontal parameter to an actual periodontal parameter from the previous periodontal parameter data, and
adjusting the initial periodontal parameter based on the comparison.

Example 70. The system of Example 69, wherein the operations further comprise using the comparison to identify an error between the predicted periodontal parameter and the actual periodontal parameter, wherein the initial periodontal parameter is adjusted to compensate for the error.

Example 71. The system of any one of Examples 67 to 70, wherein the additional data comprises one or more of demographic information, health condition data, previous periodontal parameter data, palate color data, other imaging modality data, bone height data, bone loss data, gum recession data, inflammation data, tooth mobility data, or occlusion data.

Example 72. The system of Example 71, wherein the additional data comprises the previous periodontal parameter data, and the previous periodontal parameter data comprises one or more previous periodontal pocket depth values.

Example 73. The system of Example 72, wherein the one or more previous periodontal pocket depth values are for only a subset of the patient's teeth.

Example 74. The system of Example 72, wherein the one or more previous periodontal pocket depth values are for all of the patient's teeth.

Example 75. The system of any one of Examples 71 to 74, wherein the additional data comprises the demographic information, and the demographic information comprises one or more of age, ethnicity, or gender.

Example 76. The system of any one of Examples 71 to 75, wherein the additional data comprises the other imaging modality data, and the other imaging modality data comprises x-ray data.

Example 77. The system of any one of Examples 67 to 76, wherein the scan data comprises one or more of surface topography data, color data, or near infrared image data.

Example 78. The system of any one of Examples 67 to 77, wherein the operations further comprise processing the scan data to generate a continuous texture map, prior to determining the initial periodontal parameter.

Example 79. A computer-implemented method for evaluating a patient's intraoral health, the computer-implemented method comprising:
receiving scan data of a patient's intraoral cavity;
determining, based on the scan data, an initial periodontal parameter indicative of a periodontal condition of the patient, wherein the initial periodontal parameter is determined using a machine learning model that is trained on scan data and periodontal parameter measurements from a plurality of patients;
determining whether additional data representing one or more patient-specific characteristics of the patient is available;
if the additional data is available:
   adjusting the initial periodontal parameter based on the additional data, and
   outputting an indication of the adjusted periodontal parameter on a display; and
if the additional data is not available, outputting an indication of the initial periodontal parameter on the display.

Example 80. The computer-implemented method of Example 79, wherein the additional data comprises previous scan data and previous periodontal parameter data for the patient.

Example 81. The computer-implemented method of Example 80, wherein adjusting the initial periodontal parameter comprises:
determining a predicted periodontal parameter for the patient using the machine learning model and the previous scan data,
comparing the predicted periodontal parameter to an actual periodontal parameter from the previous periodontal parameter data, and
adjusting the initial periodontal parameter based on the comparison.

Example 82. The computer-implemented method of Example 81, further comprising using the comparison to identify an error between the predicted periodontal parameter and the actual periodontal parameter, wherein the initial periodontal parameter is adjusted to compensate for the error.

Example 83. The computer-implemented method of any one of Examples 79 to 82, wherein the additional data comprises one or more of demographic information, health condition data, previous periodontal parameter data, palate color data, other imaging modality data, bone height data, bone loss data, gum recession data, inflammation data, tooth mobility data, or occlusion data.

Example 84. The computer-implemented method of Example 83, wherein the additional data comprises the previous periodontal parameter data, and the previous periodontal parameter data comprises one or more previous periodontal pocket depth values.

Example 85. The computer-implemented method of Example 84, wherein the one or more previous periodontal pocket depth values are for only a subset of the patient's teeth.

Example 86. The computer-implemented method of Example 84, wherein the one or more previous periodontal pocket depth values are for all of the patient's teeth.

Example 87. The computer-implemented method of any one of Examples 83 to 86, wherein the additional data comprises the demographic information, and the demographic information comprises one or more of age, ethnicity, or gender.

Example 88. The computer-implemented method of any one of Examples 83 to 87, wherein the additional data comprises the other imaging modality data, and the other imaging modality data comprises x-ray data.

Example 89. The computer-implemented method of any one of Examples 79 to 88, wherein the scan data comprises one or more of surface topography data, color data, or near infrared image data.

Example 90. The computer-implemented method of any one of Examples 79 to 89, further comprising processing the scan data to generate a continuous texture map, prior to determining the initial periodontal parameter.

Example 91. A system for evaluating a patient's intraoral health, the system comprising:
one or more processors; and
a memory operably coupled to the one or more processors and storing instructions that, when executed by the one or more processors, cause the system to perform operations comprising:
   receiving scan data of a patient's intraoral cavity;
   determining, based on the scan data, a periodontal pocket depth value for the patient, wherein the periodontal pocket depth value is determined using a machine learning model that is trained on scan data and periodontal parameter measurements from a plurality of patients;
   identifying a potential cause of the periodontal pocket depth value; and
   outputting an indication of the periodontal pocket depth value and the potential cause on a display.

Example 92. The system of Example 91, wherein a plurality of periodontal pocket depth values are determined for the patient, and wherein the potential cause is identified based on a number of periodontal pocket depth values that exceed a threshold value.

Example 93. The system of Example 91 or 92, wherein a plurality of periodontal pocket depth values are determined for an upper jaw and a lower jaw of the patient, and wherein the potential cause is identified based on a comparison of a number of periodontal pocket depth values in the upper jaw that exceed a threshold value and a number of periodontal pocket depth values in the lower jaw that exceed the threshold value.

Example 94. The system of any one of Examples 91 to 93, wherein the potential cause is identified based on one or more of the scan data or the periodontal pocket depth value.

Example 95. The system of any one of Examples 91 to 94, wherein the potential cause is identified based on additional data for the patient.

Example 96. The system of Example 95, wherein the additional data comprises one or more of demographic information, health condition data, previous periodontal parameter data, palate color data, other imaging modality data, bone height data, bone loss data, gum recession data, inflammation data, tooth mobility data, or occlusion data.

Example 97. The system of any one of Examples 91 to 96, wherein the potential cause comprises one or more of bruxism or inadequate brushing.

Example 98. The system of any one of Examples 91 to 97, wherein the potential cause comprises bruxism, and wherein the bruxism is identified by:

identifying a location of gingival tissue having the periodontal pocket depth value, and detecting one or more of increased tooth wear or increased occlusal contacts proximate to the identified location.

Example 99. The system of any one of Examples 91 to 98, wherein the potential cause is determined using a rule-based algorithm.

Example 100. The system of Example 99, wherein the rule-based algorithm comprises a heuristic decision tree.

Example 101. The system of any one of Examples 91 to 100, wherein the potential cause is identified using a machine learning model trained on data for a plurality of patients.

Example 102. The system of any one of Examples 91 to 101, wherein the potential cause is identified by:
detecting one or more causation factors of the patient using a first algorithm, based on one or more of the scan data, the periodontal pocket depth value, or additional data for the patient, and
identifying the potential cause using a second algorithm, based on the one or more causation factors.

Example 103. The system of Example 102, wherein the one or more causation factors comprise one or more of the following: calculus build up, increased tooth wear, increased occlusal contacts, bite force, frequency of abnormal periodontal pocket depth values, distribution of abnormal periodontal pocket depth values, blood pressure, diabetes status, or smoking status.

Example 104. The system of any one of Examples 91 to 103, wherein the operations further comprise outputting a treatment recommendation on the display, based on the potential cause.

Example 105. The system of any one of Examples 91 to 104, wherein the operations further comprise:
identifying a plurality of potential causes of the periodontal pocket depth value,
determining a confidence score associated with each potential cause, and outputting an indication of the potential causes and the associated confidence scores on the display.

Example 106. The system of any one of Examples 91 to 105, wherein the operations further comprise:
prompting a user for a user-predicted cause of the periodontal pocket depth value, and
receiving user input indicative of the user-predicted cause of the periodontal pocket depth value.

Example 107. The system of Example 106, wherein the potential cause is identified using a cause prediction algorithm, and wherein the operations further comprise:
comparing the user-predicted cause to the potential cause, and
adjusting the cause prediction algorithm based on the comparison.

Example 108. The system of Example 106 or 107, wherein the operations further comprise:
identifying a discrepancy between the user-predicted cause and the potential cause, and
storing the discrepancy in a record for the patient.

Example 109. The system of any one of Examples 91 to 108, wherein the operations further comprise:
determining whether user feedback on an accuracy of identification of a potential cause of a previous periodontal pocket depth value is available, and
if the user feedback is available, outputting an indication of the user feedback on the display.

Example 110. The system of any one of Examples 91 to 109, wherein the operations further comprise:
determining whether user feedback on an accuracy of identification of a potential cause of a previous periodontal pocket depth value is available, and
if the user feedback is available, adjusting the potential cause based on the user feedback.

Example 111. A computer-implemented method for evaluating a patient's intraoral health, the computer-implemented method comprising:
receiving scan data of a patient's intraoral cavity;
determining, based on the scan data, a periodontal pocket depth value for the patient, wherein the periodontal pocket depth value is determined using a machine learning model that is trained on scan data and periodontal parameter measurements from a plurality of patients;
identifying a potential cause of the periodontal pocket depth value; and
outputting an indication of the periodontal pocket depth value and the potential cause on a display.

Example 112. The computer-implemented method of Example 111, wherein a plurality of periodontal pocket depth values are determined for the patient, and wherein the potential cause is identified based on a number of periodontal pocket depth values that exceed a threshold value.

Example 113. The computer-implemented method of Example 111 or 112, wherein a plurality of periodontal pocket depth values are determined for an upper jaw and a lower jaw of the patient, and wherein the potential cause is identified based on a comparison of a number of periodontal pocket depth values in the upper jaw that exceed a threshold value and a number of periodontal pocket depth values in the lower jaw that exceed the threshold value.

Example 114. The computer-implemented method of any one of Examples 111 to 113, wherein the potential cause is identified based on one or more of the scan data or the periodontal pocket depth value.

Example 115. The computer-implemented method of any one of Examples 111 to 114, wherein the potential cause is identified based on additional data for the patient.

Example 116. The computer-implemented method of Example 115, wherein the additional data comprises one or more of demographic information, health condition data, previous periodontal parameter data, palate color data, other imaging modality data, bone height data, bone loss data, gum recession data, inflammation data, tooth mobility data, or occlusion data.

Example 117. The computer-implemented method of any one of Examples 111 to 116, wherein the potential cause comprises one or more of bruxism or inadequate brushing.

Example 118. The computer-implemented method of any one of Examples 111 to 117, wherein the potential cause comprises bruxism, and wherein the bruxism is identified by:
identifying a location of gingival tissue having the periodontal pocket depth value, and
detecting one or more of increased tooth wear or increased occlusal contacts proximate to the identified location.

Example 119. The computer-implemented method of any one of Examples 111 to 118, wherein the potential cause is determined using a rule-based algorithm.

Example 120. The computer-implemented method of Example 119, wherein the rule-based algorithm comprises a heuristic decision tree.

Example 121. The computer-implemented method of any one of Examples 111 to 120, wherein the potential cause is identified using a machine learning model trained on data for a plurality of patients.

Example 122. The computer-implemented method of any one of Examples 111 to 121, wherein the potential cause is identified by:
detecting one or more causation factors of the patient using a first algorithm, based on one or more of the scan data, the periodontal pocket depth value, or additional data for the patient, and
identifying the potential cause using a second algorithm, based on the one or more causation factors.

Example 123. The computer-implemented method of Example 122, wherein the one or more causation factors comprise one or more of the following: calculus build up, increased tooth wear, increased occlusal contacts, bite force, frequency of abnormal periodontal pocket depth values, distribution of abnormal periodontal pocket depth values, blood pressure, diabetes status, or smoking status.

Example 124. The computer-implemented method of any one of Examples 111 to 123, further comprising outputting a treatment recommendation on the display, based on the potential cause.

Example 125. The computer-implemented method of any one of Examples 111 to 124, further comprising:
identifying a plurality of potential causes of the periodontal pocket depth value,
determining a confidence score associated with each potential cause, and
outputting an indication of the potential causes and the associated confidence scores on the display.

Example 126. The computer-implemented method of any one of Examples 111 to 125, further comprising:
prompting a user for a user-predicted cause of the periodontal pocket depth value, and
receiving user input indicative of the user-predicted cause of the periodontal pocket depth value.

Example 127. The computer-implemented method of Example 116, wherein the potential cause is identified using a cause prediction algorithm, and wherein the computer-implemented method further comprises:
comparing the user-predicted cause to the potential cause, and
adjusting the cause prediction algorithm based on the comparison.

Example 128. The computer-implemented method of Example 116 or 117, further comprising:
identifying a discrepancy between the user-predicted cause and the potential cause, and
storing the discrepancy in a record for the patient.

Example 129. The computer-implemented method of any one of Examples 111 to 128, further comprising:
determining whether user feedback on an accuracy of identification of a potential cause of a previous periodontal pocket depth value is available, and
if the user feedback is available, outputting an indication of the user feedback on the display.

Example 130. The computer-implemented method of any one of Examples 111 to 129, further comprising:
determining whether user feedback on an accuracy of identification of a potential cause of a previous periodontal pocket depth value is available, and
if the user feedback is available, adjusting the potential cause based on the user feedback.

Example 131. A system for evaluating a patient's intraoral health, the system comprising:
one or more processors; and
a memory operably coupled to the one or more processors and storing instructions that, when executed by the one or more processors, cause the system to perform operations comprising:
   receiving scan data of a patient's intraoral cavity;
   receiving additional data for the patient, the additional data being associated with a different measurement modality than the scan data;
   detecting, based on the scan data and the additional data, a condition of the patient's intraoral cavity; and
   outputting an indication of the detected condition on a display.

Example 132. The system of Example 131, wherein the condition comprises one or more of a periodontal disease, a deep periodontal pocket, bruxism, tooth decay, or dental plaque.

Example 133. The system of Example 131 or 132, wherein the additional data comprises one or more of x-ray data, bone height data, bone loss data, gum recession data, inflammation data, tooth mobility data, or occlusion data.

Example 134. The system of any one of Examples 131 to 133, wherein the detecting is performed by inputting the scan data and the additional data into a machine learning model, and wherein the machine learning model is trained on scan data, additional data, and condition data from a plurality of patients.

Example 135. The system of Example 134, wherein the operations further comprise:
generating an input data set by combining the scan data and the additional data, and
inputting the input data set into the machine learning model.

Example 136. The system of any one of Examples 131 to 133, wherein the detecting is performed by:
generating a first prediction and a first confidence score for the first prediction by inputting the scan data into a first algorithm,
generating a second prediction and a second confidence score for the second prediction by inputting the additional data into a second algorithm, and
determining the condition of the patient's intraoral cavity based on the first prediction, the first confidence score, the second prediction, and the second confidence score.

Example 137. The system of Example 136, wherein the operations further comprise determining whether at least one of the first or second confidence scores exceeds a threshold value.

Example 138. The system of Example 136 or 137, wherein the first algorithm comprises a machine learning model trained on scan data and condition data from a plurality of patients.

Example 139. The system of any one of Examples 136 to 138, wherein the first algorithm comprises a rule-based algorithm.

Example 140. The system of any one of Examples 136 to 139, wherein the second algorithm comprises a machine learning model trained on additional data and condition data from a plurality of patients.

Example 141. The system of any one of Examples 136 to 140, wherein the second algorithm comprises a rule-based algorithm.

Example 142. A computer-implemented method for evaluating a patient's intraoral health, the computer-implemented method comprising:
receiving scan data of a patient's intraoral cavity;
receiving additional data for the patient, the additional data being associated with a different measurement modality than the scan data;
detecting, based on the scan data and the additional data, a condition of the patient's intraoral cavity; and
outputting an indication of the detected condition on a display.

Example 143. The computer-implemented method of Example 142, wherein the condition comprises one or more of a periodontal disease, a deep periodontal pocket, bruxism, tooth decay, or dental plaque.

Example 144. The computer-implemented method of Example 142 or 143, wherein the additional data comprises one or more of x-ray data, bone height data, bone loss data, gum recession data, inflammation data, tooth mobility data, or occlusion data.

Example 145. The computer-implemented method of any one of Examples 142 to 144, wherein the detecting is performed by inputting the scan data and the additional data into a machine learning model, and wherein the machine learning model is trained on scan data, additional data, and condition data from a plurality of patients.

Example 146. The computer-implemented method of Example 145, further comprising:
generating an input data set by combining the scan data and the additional data, and
inputting the input data set into the machine learning model.

Example 147. The computer-implemented method of any one of Examples 142 to 144, wherein the detecting is performed by:
generating a first prediction and a first confidence score for the first prediction by inputting the scan data into a first algorithm,
generating a second prediction and a second confidence score for the second prediction by inputting the additional data into a second algorithm, and
determining the condition of the patient's intraoral cavity based on the first prediction, the first confidence score, the second prediction, and the second confidence score.

Example 148. The computer-implemented method of Example 147, further comprising determining whether at least one of the first or second confidence scores exceeds a threshold value.

Example 149. The computer-implemented method of Example 147 or 148, wherein the first algorithm comprises a machine learning model trained on scan data and condition data from a plurality of patients.

Example 150. The computer-implemented method of any one of Examples 147 to 149, wherein the first algorithm comprises a rule-based algorithm.

Example 151. The computer-implemented method of any one of Examples 147 to 150, wherein the second algorithm comprises a machine learning model trained on additional data and condition data from a plurality of patients.

Example 152. The computer-implemented method of any one of Examples 147 to 151, wherein the second algorithm comprises a rule-based algorithm.

Example 153. A non-transitory computer-readable storage medium comprising instructions that, when executed by one or more processors of a computing system, cause the computing system to perform operations comprising the computer-implemented method of any one of Examples 11 to 20, 26 to 30, 49 to 65, 79 to 90, 111 to 130, or 142 to 152.

Example 154. The system of any one of the preceding Examples, wherein the operations comprise receiving intraoral scan data and determining one or more periodontal pocket depth values based on the intraoral scan data.

Example 155. The computer-implemented method of any one of the preceding Examples, comprising receiving intraoral scan data and determining one or more periodontal pocket depth values based on the intraoral scan data.

### CONCLUSION

Although many of the embodiments are described above with respect to systems, devices, and methods for predicting periodontal pocket depths, the technology is applicable to other applications and/or other approaches, such as predicting other intraoral conditions and/or conditions of other anatomical regions. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-15B.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A system for evaluating a patient's intraoral health, the system comprising:
one or more processors; and
a memory operably coupled to the one or more processors and storing instructions that, when executed by the one or more processors, cause the system to perform operations comprising:
receiving scan data of a patient's intraoral cavity;
receiving additional data for the patient, the additional data being different from the scan data;
determining a first prediction of a condition of the intraoral cavity based on the scan data;
determining a second prediction of a condition of the intraoral cavity based on the additional data;
generating a predicted condition for the intraoral cavity based on a combination of the first and second predictions; and
outputting an indication of the predicted condition on a display.

2. The system of claim 1, wherein the additional data comprises one or more of demographic information, health condition data, previous periodontal parameter data, palate color data, other imaging modality data, bone height data, bone loss data, gum recession data, inflammation data, tooth mobility data, or occlusion data.

3. The system of claim 1, wherein the first prediction comprises a periodontal parameter, and the second prediction comprises an intraoral parameter different from the periodontal parameter.

4. The system of claim 3, wherein the periodontal parameter comprises a periodontal pocket depth value.

5. The system of claim 3, wherein the intraoral parameter is indicative of one or more of tooth mobility, gum recession, inflammation, bone height, or bone loss.

6. The system of claim 1, wherein the predicted condition comprises an oral health metric.

7. The system of claim 6, wherein the oral health metric is representative of overall oral health for a plurality of teeth.

8. The system of claim 1, wherein the first prediction is determined using a first condition prediction algorithm, and wherein the second prediction is determined using a second condition prediction algorithm different from the first condition prediction algorithm.

9. The system of claim 1, wherein the indication is displayed together with a digital representation of the patient's teeth.

10. The system of claim 1, wherein the operations further comprise outputting a treatment recommendation on the display, and wherein the treatment recommendation comprises performing an additional diagnostic procedure at a location associated with the predicted condition.

11. A system for evaluating a patient's intraoral health, the system comprising:
one or more processors; and
a memory operably coupled to the one or more processors and storing instructions that, when executed by the one or more processors, cause the system to perform operations comprising:
determining, based on scan data of a patient's intraoral cavity, a periodontal parameter for one or more teeth of the intraoral cavity;
determining, based on additional data for the patient, an intraoral parameter for the one or more teeth, the intraoral parameter being different from the periodontal parameter;
generating based on the periodontal parameter and the intraoral parameter, an oral health metric for the one or more teeth;
outputting, on a display device, the oral health metric together with a digital representation of the one or more teeth; and
outputting, on the display device, in response to user input selecting the oral health metric, one or more of the periodontal parameter or the intraoral parameter.

12. The system of claim 11, wherein the additional data comprises one or more of demographic information, health condition data, previous periodontal parameter data, palate color data, other imaging modality data, bone height data, bone loss data, gum recession data, inflammation data, tooth mobility data, or occlusion data.

13. The system of claim 11, wherein the periodontal parameter comprises a periodontal pocket depth value.

14. The system of claim 11, wherein the intraoral parameter is indicative of one or more of tooth mobility, gum recession, inflammation, bone height, or bone loss.

15. The system of claim 11, wherein the oral health metric is representative of overall oral health for a plurality of teeth.

16. A system for evaluating a patient's intraoral health, the system comprising:
one or more processors; and
a memory operably coupled to the one or more processors and storing instructions that, when executed by the one or more processors, cause the system to perform operations comprising:
receiving scan data of a patient's intraoral cavity;
receiving additional data for the patient, the additional data being associated with a different measurement modality than the scan data;
detecting, based on the scan data and the additional data, a condition of the patient's intraoral cavity; and
outputting an indication of the detected condition on a display.

17. The system of claim 16, wherein the condition comprises one or more of a periodontal disease, a deep periodontal pocket, bruxism, tooth decay, or dental plaque.

18. The system of claim 16, wherein the additional data comprises one or more of x-ray data, bone height data, bone loss data, gum recession data, inflammation data, tooth mobility data, or occlusion data.

19. The system of claim 16, wherein the detecting is performed by:
generating a first prediction by inputting the scan data into a first algorithm,
generating a second prediction by inputting the additional data into a second algorithm, and
determining the condition of the patient's intraoral cavity based on the first prediction and the second prediction.

20. The system of claim 19, wherein the first algorithm comprises a machine learning model trained on scan data and condition data from a plurality of patients, and wherein the second algorithm comprises a machine learning model trained on additional data and condition data from a plurality of patients.
